(19) United States
(12) Patent Application Publication
(10) Pub. No.: US 2024/0368695 A1
(43) Pub. Date: Nov. 7, 2024

(54) EMBRYONIC NUCLEIC ACID ANALYSIS

(71) Applicant: BioSkryb Genomics, Inc., Durham, NC (US)

(72) Inventors: JAY A.A. WEST, Durham, NC (US); Jon ZAWISTOWSKI, Durham, NC (US)

(21) Appl. No.: 18/683,925

(22) PCT Filed: Aug. 15, 2022
(86) PCT No.: PCT/US2022/040325

§ 371 Date: Feb. 15, 2024

Related U.S. Application Data
(60) Provisional application No. 63/233,662, filed on Aug. 16, 2021.

(51) Int. Cl. C12Q 1/6883; C12Q 1/6806

(52) U.S. Cl. CPC C12Q 1/6883; C12Q 1/6806; C12Q 2600/156

(57) ABSTRACT

Provided herein are compositions and methods for high-throughput Primary Template-Directed Amplification (PTA) nucleic acid amplification and sequencing methods, and their applications for mutational analysis of embryonic cells. Further provided are methods of simultaneous determination of genetic abnormalities in embryonic cells, wherein the fetal genetic abnormalities comprise: i. at least one copy number variation; and ii. at least one single nucleotide variant.

### CROSS-REFERENCE

This application claims the benefit of U.S. Provisional Application No. 63/233,662, filed Aug. 16, 2021, which application is incorporated herein by reference.

### BACKGROUND

Research methods that utilize nucleic amplification, e.g., Next Generation Sequencing, provide large amounts of information on complex samples, genomes, and other nucleic acid sources. In particular such methods may be used for analysis of fetal samples for abnormalities. However, some methods of analysis require multiple workflows that suffer from low efficiency or accuracy, and must sample different groups of cells for each method. There exists a need for highly accurate, scalable, and efficient nucleic acid amplification and sequencing methods for analysis of these samples.

### SUMMARY

In certain aspects, described herein is a method of embryonic nucleic acid sample preparation useful for determining the presence of fetal genetic abnormalities comprising: isolating nucleic acids from at least one embryonic cell; subjecting the nucleic acids to a sample workflow; and determining if the embryonic cell comprises at least two fetal genetic abnormalities by analyzing the nucleic acids from the sample workflow, wherein the fetal genetic abnormalities comprise: at least one copy number variation; and at least one single nucleotide variant. In some embodiments, the embryonic cell comprises a preimplantation embryonic cell, a blastocyte cell, blastomere cell, a cell obtained from the trophectoderm, a placental cell, or a cell derived from extra-embryonic membranes. In some embodiments, the embryonic cell comprises a preimplantation embryonic cell. In some embodiments, the fetal genetic abnormality comprises two or more of aneuploidy, monogenic disorders, and structural rearrangements. In some embodiments, the fetal genetic abnormality comprises aneuploidy, monogenic disorders, and structural rearrangements. In some embodiments, determining comprises obtaining information on fetal genetic abnormalities identifiable by PGT-A, PGT-M, or PGT-SR testing. In some embodiments, the genetic abnormality comprises phenylketonuria (PKU), sickle-cell anemia, Beta Thalassemia, Tay-Sachs disease, Sandhoff disease, or cystic fibrosis (CF). In some embodiments, the genetic abnormality comprises achondroplasia, congenital adrenal hyperplasia, Cystic fibrosis, Down syndrome, fragile XD syndrome, Hemophilia A, Huntington's disease, Muscular dystrophy, Polycystic kidney disease, Sickle cell disease, Tay-Sachs disease, trisomy 21, trisomy 18, trisomy 13, Turner syndrome, spina bifida, anencephaly, or Thalassemia. In some embodiments, the aneuploidy comprises monosomy, trisomy, triploidy, deletions, duplications, or uniparental disomy. In some embodiments, the uniparental disomy occurs at least in four chromosomes. In some embodiments, the uniparental disomy occurs at chromosomes 6, 7, 11, 14, or 15. In some embodiments, the fetal genetic abnormality comprises an insertion, deletion or duplication. In some embodiments, the insertion, deletion or duplication is at least 5% of the total chromosome length. In some embodiments, the insertion, deletion, or duplication is less than 15% of the total chromosome length. In some embodiments, the method further comprises obtaining the cell from at least a 5 day old blastocyte. In some embodiments, the method comprises obtaining at least 4 embryonic cells. In some embodiments, a fetal genetic abnormality is detected in no more than 30% of the embryonic cells. In some embodiments, a fetal genetic abnormality is detected in 30%-100% of the embryonic cells. In some embodiments, the method further comprises obtaining the embryonic cell from a location proximal to an external os of a uterine cervix or anywhere within the vaginal canal of a subject. In some embodiments, the method further comprises obtaining the embryonic cell from a Pap smear. In some embodiments, the embryonic cell is human. In some embodiments, the method comprises obtaining 6-200 embryonic cells. In some embodiments, the method further comprises measuring a level of mosaicism for the embryonic cells. n some embodiments, the method further comprises establishing the presence or absence of sex chromosomes in the embryonic cell. In some embodiments, the embryonic cell is a preimplantation embryonic cell from an embryo, and the method further comprises implanting the embryo in a female. In some embodiments, the fetal genetic abnormalities are determined without a blood or saliva test. In some embodiments, the fetal genetic abnormalities are determined without amniocentesis, chorionic villus sampling, or Percutaneous umbilical blood sampling. In some embodiments, determining comprises sequencing the nucleic acids. In some embodiments, sequencing comprises Sanger sequencing, next generation sequencing, single-molecule real-time sequencing, Polony sequencing, sequencing by synthesis, sequencing by ligation, reversible terminator sequencing, proton detection sequencing, ion semiconductor sequencing, nanopore sequencing, electronic sequencing, pyrosequencing, Maxam-Gilbert sequencing, chain termination sequencing, or +S sequencing. In some embodiments, the method further comprises exome capture prior to sequencing. In some embodiments, the sample workflow comprises: contacting the nucleic acids with at least one amplification primer, at least one nucleic acid polymerase, and a mixture of nucleotides, wherein the mixture of nucleotides comprises at least one terminator nucleotide which terminates nucleic acid replication by the polymerase, and amplifying at least some of the nucleic acids to generate a plurality of terminated amplification products, wherein the replication proceeds by strand displacement replication.

In certain aspects, described herein is a method of embryonic nucleic acid sample preparation useful for determining the presence of fetal genetic abnormalities comprising: isolating at least one embryonic cell from a plurality of cells, wherein the plurality of cells comprises fetal and maternal cells; isolating nucleic acids from the at least one embryonic cell; contacting the nucleic acids with at least one amplification primer, at least one nucleic acid polymerase, and a mixture of nucleotides, wherein the mixture of nucleotides comprises at least one terminator nucleotide which terminates nucleic acid replication by the polymerase; amplifying at least some of the nucleic acids to generate a plurality of terminated amplification products, wherein the replication proceeds by strand displacement replication; and determining if the embryonic cell comprises one or more genetic abnormalities by analyzing the terminated amplification products. In some embodiments, the embryonic cell is isolated from the plurality of cells on a surface. In some embodiments, the fetal cells are obtained from one or more of the trophectoderm, placenta, or extra-embryonic membranes. In some embodiments, the embryonic cell is isolated by an automated robotic device. In some embodiments, the robotic device comprises a capillary fitting. In some embodiments, the embryonic cell is uniquely identified from other non-embryonic cells. In some embodiments, the embryonic cell is uniquely identified from other non-embryonic cells by labeling. In some embodiments, labeling comprises contacting the plurality of cells with an antibody. In some embodiments, the antibody is configured to bind selectively to non-fetal cells. In some embodiments, the antibody is configured to bind selectively to fetal cells. In some embodiments, the antibody is configured to bind to HLA-G or βhCG. In some embodiments, labeling comprises Hoescht staining. In some embodiments, isolating comprises FACS sorting. In some embodiments, the antibody comprises a magnetic nanoparticle. In some embodiments, the embryonic cell is removed as early as 5 weeks after pregnancy. In some embodiments, the embryonic cell is removed as early as 8 weeks after pregnancy. In some embodiments, determining comprises in-silico removal of maternal nucleic acid sequences. In some embodiments, the terminator is an irreversible terminator. In some embodiments, the terminator nucleotide is selected from the group consisting of nucleotides with modification to the alpha group, C3 spacer nucleotides, locked nucleic acids (LNA), inverted nucleic acids, 2′ fluoro nucleotides, 3′ phosphorylated nucleotides, 2′-O-Methyl modified nucleotides, and trans nucleic acids. In some embodiments, the nucleotides with modification to the alpha group are alpha-thio dideoxynucleotides. In some embodiments, the terminator nucleotide comprises modifications of the r group of the 3′ carbon of the deoxyribose. In some embodiments, the terminator nucleotide is selected from the group consisting of dideoxynucleotides, inverted dideoxynucleotides, 3′ biotinylated nucleotides, 3′ amino nucleotides, 3′-phosphorylated nucleotides, 3′-O-methyl nucleotides, 3′ carbon spacer nucleotides including 3′ C3 spacer nucleotides, 3′ C18 nucleotides, 3′ Hexanediol spacer nucleotides, acyclonucleotides, and combinations thereof. In some embodiments, the plurality of terminated amplification products comprise an average of 1000-2000 bases in length. In some embodiments, at least some of the amplification products comprise a cell barcode or a sample barcode. In some embodiments, amplification occurs for at least five cycles.

In certain aspects, described herein is a method of embryonic nucleic acid sample preparation useful for determining the presence or absence of fetal genetic abnormalities comprising: obtaining a plurality of embryonic cells, wherein the plurality of embryonic cells comprises between 2 and 200 embryonic cells; isolating nucleic acids from the plurality of embryonic cells; contacting the nucleic acids with at least one amplification primer, at least one nucleic acid polymerase, and a mixture of nucleotides, wherein the mixture of nucleotides comprises at least one terminator nucleotide which terminates nucleic acid replication by the polymerase; amplifying at least some of the nucleic acids to generate a plurality of terminated amplification products, wherein the replication proceeds by strand displacement replication; and determining if the plurality of embryonic cells comprises one or more genetic abnormalities by analyzing the terminated amplification products. In some embodiments, the fetal cells are obtained from one or more of the trophectoderm, placenta, or extra-embryonic membranes

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A illustrates a plot of yield for various amounts of template (1 ng-10 pg) or single cells (SC1-SC8) for a Primary Template-Directed Amplification (PTA) reaction.

FIG. 1B illustrates a plot of amplicon sizes after PTA.

FIG. 1C illustrates a plot of amplicon sizes after library generation from PTA-generated amplicons.

FIG. 1D illustrates a plot of sensitivity vs. precision of SNV calling in GM12878 single cells.

FIG. 2 illustrates a workflow for analysis of embryonic cells obtained for either pre-implantation testing or Pap smear. Methods described herein allow for simultaneous aneuploidy detection (CNV/PGTa), targeted mutation detection (PGTm), and aneuploidy detection CNV/PGTa/m/SR).

FIG. 3 illustrates a copy number profile for aneuploidy. Data points are shown at 2.5 million base pair intervals. Trisomy was observed at chromosome 21.

FIG. 4 illustrates a copy number profile showing multiple CNVs. Data points are shown at 2.5 million base pair intervals.

FIG. 5 depicts wells containing 1-200 FACs sorted cells for use in downstream analysis.

FIG. 6 depicts yields of PTA amplified DNA from samples of 1-200 sorted cells.

FIG. 7 depicts low pass sequencing metrics of 1 cell, 5 cell, 10 cell, 20 cell, 50 cell, 75 cell, 100 cell and 200 cell reactions.

FIG. 8 depicts low pass sequencing metrics for a round of sequencing of embryonic cells.

FIG. 9 depicts low pass sequencing metrics for a round of sequencing of embryonic cells.

FIG. 10 depicts low pass sequencing metrics for a round of sequencing of embryonic cells.

FIG. 11A represents an experimental workflow.

FIG. 11B depicts light field (left panels) and fluorescent (right panels) images of cells stained with Hoescht staining, as well as unstained cells.

FIG. 11C depicts individual yields of PTA amplified DNA from Hoescht stained cells and unstained cells.

FIG. 11D depicts the average yield of PTA amplified DNA from Hoescht stained cells and unstained cells.

FIG. 12 depicts the PGT integrated workflow enabling streamlined PGT-A and PGT-M workflows from an individual embryo cell.

FIG. 13 depicts Genome coverage summary for performance on embryo samples. Genome coverage (x-axis) and average genome depth (y-axis) shown. Roughly 78 Gbp were generated for 20× depth.

FIG. 14 depicts the allelic balance of embryo samples through the workflow.

FIG. 15 depicts examples of output from PGT-A copy number variation (CNV) calling from PGT workflow.

### DETAILED DESCRIPTION OF THE INVENTION

There is a need to develop new scalable, accurate and efficient methods for nucleic acid amplification (including nucleic acids from fetal cells) and sequencing which would overcome limitations in the current methods by increasing sequence representation, uniformity and accuracy in a reproducible manner. Provided herein are compositions and methods for providing accurate and scalable Primary Template-Directed Amplification (PTA) and sequencing. Further provided herein are methods of analyzing nucleic acids from fetal cells with PTA. Further provided herein are methods of using PTA for pre-implantation or non-invasive genetic testing. Further provided herein are methods of determining fetal genetic abnormalities or other characteristics using PTA.

### Primary Template-Directed Amplification (PTA) of Fetal Genetics

Provided herein are methods for analysis of nucleic acids using PTA. In some instances, nucleic acids are obtained from embryonic cells or other cells related to fetal development. In some instances, methods comprise analyzing fetal cells for genetic abnormalities. In some instances, a single workflow provides information for diagnosing multiple genetic abnormalities. In some instances, methods described herein comprise analysis of human fetal cells.

Provided herein are methods for preparing embryonic nucleic acids. In some instances, the method comprises PTA. In some instances, methods comprise one or more of isolating nucleic acids from at least one cell; subjecting the nucleic acids to a sample workflow; and determining if the cell comprises a genetic abnormality by analyzing the nucleic acids from the sample workflow. In some instances, method comprise one or more of isolating nucleic acids from at least one embryonic cell; subjecting the nucleic acids to a sample workflow; and determining if the embryonic cell comprises at least two fetal genetic abnormalities by analyzing the nucleic acids from the sample workflow. In some instances, the fetal genetic abnormalities comprise: at least one copy number variation or at least one single nucleotide variant. In some instances, the fetal genetic abnormalities comprise: at least one copy number variation and at least one single nucleotide variant. In some instances, at least two, three, four, five, or more genetic abnormalities are determined. In some instances, methods described herein further comprise contacting the nucleic acids with at least one amplification primer, at least one nucleic acid polymerase, and a mixture of nucleotides, wherein the mixture of nucleotides comprises at least one terminator nucleotide which terminates nucleic acid replication by the polymerase, and amplifying at least some of the nucleic acids to generate a plurality of terminated amplification products, wherein the replication proceeds by strand displacement replication. In some instances, determining comprises sequencing methods described herein. In some instances, the terminated amplification products are converted into sequencing-ready libraries. In some instances, terminated amplification products are captured, enriched, or selected for specific sequences of interest. In some instances, sequences of interest comprise exons.

Provided herein are methods for preparing a fetal cell sample useful for determining the presence or absence of genetic abnormalities. In some instances, the method comprises PTA. In some instances, the method comprises one or more of isolating a cell from a plurality of cells; isolating nucleic acids from the embryonic cell; contacting the nucleic acids with at least one amplification primer, at least one nucleic acid polymerase, and a mixture of nucleotides, wherein the mixture of nucleotides comprises at least one terminator nucleotide which terminates nucleic acid replication by the polymerase, and amplifying at least some of the nucleic acids to generate a plurality of terminated amplification products, wherein the replication proceeds by strand displacement replication and determining if the cell comprises one or more genetic abnormalities by analyzing the terminated amplification products. In some instances, cells comprise fetal cells. In some instances, the plurality of cells comprises fetal and maternal cells. In some instances, the terminated amplification products are converted into sequencing-ready libraries. In some instances, terminated amplification products are captured, enriched, or selected for specific sequences of interest. In some instances, sequences of interest comprise exons. In some instances, cells are obtained from an endocervical sample. In some instances, cells are obtained from a Pap smear. In some instances, fetal cells are obtained from a trophoblast.

Nucleic acids may be obtained from fetal cells. In some instances, fetal cells include cells from any stage of fetal development. In some instances, fetal cells comprise embryonic cells. In some instances, embryonic cells are obtained from biopsy of an embryo. In some instances, embryonic cells are obtained from a pre-implantation embryo. In some instances, embryonic cells are obtained from a pregnant female after implantation of the embryo.

Cells may be obtained (or sampled) from fetal cells before implantation in a host (e.g., in vitro-fertilization). Fetal cells may be obtained from any variety of mammals, including humans, dogs, cats, pigs, horses, cows, goats, monkeys, rats, mice, rabbits, or other mammal. In some instances, analysis for genetic abnormalities prior to implantation of embryos provides information on the heath and viability of the embryo. In some instances, embryos with genetic abnormalities are not implanted. In some instances, cells sampled from a single embryo are compared for genetic differences (i.e., mosaicism). In some instances, embryos with lower mosaicism (more genetically heterogeneous) are implanted in the host. In some instances, methods described herein measure a level of mosaicism for an embryo. In some instances, the level of mosaicism is measured as a percentage of sampled embryonic cells which comprise one more genetic abnormalities. In some instances, at least 5%, 10%, 20%, 30%, 40%, 50%, 60%, 70%, 80%, 90%, or at least 100% of the embryonic cells comprise one more genetic abnormalities. In some instances, at no more than 5%, 10%, 20%, 30%, 40%, 50%, 60%, 70%, 80%, 90%, or no more than 100% of the embryonic cells comprise one more genetic abnormalities. In some instances, mosaicism is categorized by the number of cells comprising one more genetic abnormalities. In some instances mosaicism is categorized as uniform aneuploidies (100% of cells have mutation), high (50-70%), low (30-50%), or segmental (duplications/deletions >10 Mb). In some instances, about 5%, 10%, 20%, 30%, 40%, 50%, 60%, 70%, 80%, 90%, or about 100% of the embryonic cells comprise one more genetic abnormalities. In some instances, 1%-100%, 1%-80%, 1%-50%, 3%-30%, 5%-50%, 10%-100%, 10%-50%, 25%-100%, 30%-100%, 30%-80%, 30%-75%, 30%-50%, 50%-70%, or 50%-95% the embryonic cells comprise one more genetic abnormalities. In some instances, a fetal cell comprises preimplantation embryonic cell, a blastocyte cell, trophoblast cell, blastomere cell, a cell obtained from the trophectoderm, or a placental cell. In some instances, fetal cells are obtained from a pre-implantation embryo. In some instances, fetal cells are sampled from the embryo, and one or more of the sampled cells are analyzed using the PTA method. In some instances, no more than 20%, 15%, 10%, 7%, 5%, 3%, 2%, or no more than 1% of the pre-implantation embryo cells are sampled. In some instances, about 20%, 15%, 10%, 7%, 5%, 3%, 2%, or about 1% of the pre-implantation embryo cells are sampled. In some instances, 1%-15%, 1%-12%, 1%-10%, 1%-7%, 1%-5%, 1%-3%, 2%-10%, 2%-15%, 3%-20%, 5%-20%, 8%-20% 10%-20% of the pre-implantation embryo cells are sampled. In some instances, embryonic cells are obtained from a 1, 2, 3, 4, 5, 6, 7, 8, 9, 10, 15, 20, or 25 day old embryo. In some instances, embryonic cells are obtained from at least a 1, 2, 3, 4, 5, 6, 7, 8, 9, or at least a 10 day old embryo. In some instances, embryonic cells are obtained from an embryo that is no more than 1, 2, 3, 4, 5, 6, 7, 8, 9, or no more than 10 days old. In some instances, at least 1, 2, 3, 4, 5, 6, 7, 8, 9, 10, 15, 20, or at least 25 cells are sampled from the preimplantation embryo. In some instances about 1, 2, 3, 4, 5, 6, 7, 8, 9, 10, 15, 20, or about 25 cells are sampled from the preimplantation embryo. In some instances, no more than 1, 2, 3, 4, 5, 6, 7, 8, 9, 10, 15, 20, or no more than 25 cells are sampled from the preimplantation embryo.

Embryonic cells may be obtained after the embryo is implanted in a host. In some instances, embryonic cells are obtained from an endocervical sample. In some instances, an endocervical sample comprises cells collected from the endocervical canal. In some instances, the endocervical sample is from a pregnant subject. In some instances, fetal cells are obtained using a Pap smear. In some instances, the endocervical sample comprises maternal cells and/or fetal cells. In some in instances, fetal cells or fetal nucleic acids thereof are separated from maternal cells before analysis. In some in instances, fetal cells or nucleic acids thereof are separated physically from maternal cells before analysis. In some in instances, fetal cells or nucleic acids thereof are separated in-silico from maternal cells before or during analysis. After separation, in some instances the nucleic acids comprise at least 5-6% fetal DNA, 7-8% fetal DNA, 9-10% fetal DNA, 11-12% fetal DNA, 13-14% fetal DNA. 15-16% fetal DNA, 16-17% fetal DNA, 17-18% fetal DNA, 18-19% fetal DNA, 19-20% fetal DNA, 20-21% fetal DNA, 21-22% fetal DNA, 22-23% fetal DNA, 23-24% fetal DNA, 24-25% fetal DNA, 25-35% fetal DNA, 35-45% fetal DNA, 45-55% fetal DNA, 55-65% fetal DNA, 65-75% fetal DNA, 75-85% fetal DNA, 85-90% fetal DNA, 90-91% fetal DNA, 91-92% fetal DNA, 92-93% fetal DNA, 93-94% fetal DNA, 94-95% fetal DNA, 95-96% fetal DNA, 96-97% fetal DNA, 97-98% fetal DNA, 98-99% fetal DNA, or 99-99.7% fetal DNA. In some instances, methods described herein sample at least 1, 2, 5, 10, 15, 20, 25, 30, 50, 60, 70, 100, 125, 150, 175, 200, 225, 250, 300, 400, 500, or at least 600 cells. In some instances, methods described herein sample at least 1, 2, 5, 10, 15, 20, 25, 30, 50, 60, 70, 100, 125, 150, 175, 200, 225, 250, 300, 400, 500, or at least 600 fetal cells. In some instances, methods described herein sample no more than 1, 2, 5, 10, 15, 20, 25, 30, 50, 60, 70, 100, 125, 150, 175, 200, 225, 250, 300, 400, 500, or no more than 600 cells. In some instances, methods described herein sample no more than 1, 2, 5, 10, 15, 20, 25, 30, 50, 60, 70, 100, 125, 150, 175, 200, 225, 250, 300, 400, 500, or no more than 600 fetal cells. In some instances, methods described herein sample 1-500, 1-400, 1-300, 1-200, 1-100, 1-50, 1-25, 5-500, 5-300, 5-200, 5-100, 6-500, 6-300, 6-200, 6-100, 10-500, 10-300, 10-200, 25-300, 50-300, or 100-300 fetal cells. In some instances, methods described herein sample 1-500, 1-400, 1-300, 1-200, 1-100, 1-50, 1-25, 5-500, 5-300, 5-200, 5-100, 6-500, 6-300, 6-200, 6-100, 10-500, 10-300, 10-200, 25-300, 50-300, or 100-300 cells. Embryonic cells in some instances are obtained at least 1, 2, 3, 4, 5, 6, 7, 8, 9, 10, or at least 12 weeks after pregnancy. Embryonic cells in some instances are obtained no later than 1, 2, 3, 4, 5, 6, 7, 8, 9, 10, or no later than 12 weeks after pregnancy. Embryonic cells in some instances are obtained about 1, 2, 3, 4, 5, 6, 7, 8, 9, 10, or about 12 weeks after pregnancy. Embryonic cells in some instances are obtained about 1-12, 1-10, 1-5, 2-10, 2-8, 3-8, 4-8, 5-8, 6-8, or 6-10 weeks after pregnancy.

Cells obtained from a pregnant female may be separated from a larger population of cells. In some instances, cells are separated, isolated, labeled, or otherwise sorted based on being maternal or fetal cells. In some instances, cells are obtained in a solution. In some instances, cells are spread on a surface. In some instances, the surface comprises a plate or slide. In some instances, cells are isolated by an automated robotic device. In some instances, the robotic device comprises a capillary fitting. In some instances, the cell is uniquely identified from other non-embryonic cells. In some instances, the cell is uniquely identified from other non-embryonic cells by labeling. In some instances, cells are labeled with a label comprising a fluorophore, affinity tags, magnetic particle or other label method known in the art. In some instances, labeling comprises contacting the plurality of cells with a small molecule, antibody, antibody conjugate, or fragment thereof. In some instances, the antibody is configured to bind selectively to non-fetal cells. In some instances, the antibody is configured to bind selectively to fetal cells. In some instances, antibody is configured to bind to HLA-G or βhCG. In some embodiments, the labeling is a nuclear label. In some embodiments, the labeling is Hoescht staining. In some instances, isolating comprises FACS sorting. In some instances, the antibody comprises a magnetic nanoparticle. In some instances, maternal nucleic acid sequences are removed or filtered in-silico after sequencing.

### Genetic Analysis

Provided herein are methods of genetic analysis using PTA. In some instances, methods determine if a cell (e.g., fetal cell) comprises a genetic abnormality. In some instances, the methods described herein provide non-abnormal genetic information, such as the sex of the embryo. In some instances, the methods described herein establish the presence or absence of sex chromosomes. In some instances, the genetic abnormality includes aneuploidy, monogenic disorders, and structural rearrangements. In some instances, genetic analysis is conducted on pre-implantation embryonic cells. In some instances, genetic analysis comprises one or more of PGT-A, PGT-M, and PGT-SR genetic tests. In some instances, the genetic abnormality comprises aneuploidy. In some instances, aneuploidy comprises monosomy, trisomy, triploidy, deletions, duplications, or uniparental disomy. In some instances, aneuploidy occurs at least 1, 2, 3, 4, 5, 6, 7, 8, 9, or at least 10 chromosomes. In some instances, aneuploidy occurs in about 1, 2, 3, 4, 5, 6, 7, 8, 9, or about 10 chromosomes. In some instances, aneuploidy occurs in no more than 1, 2, 3, 4, 5, 6, 7, 8, 9, or no more than 10 chromosomes. In some instances, aneuploidy occurs at one or more of chromosomes 1, 2, 3, 4, 5, 6, 7, 8, 9, 10, 11, 12, 13, 14, 15, 16, 17, 18, 19, 20, 21, 22, or 23. In some instances aneuploidy occurs at one or more of chromosomes 13, 18, or 21. In some instances aneuploidy occurs at one or more of chromosomes 6, 7, 11, 14, or 15. In some instances, the genetic abnormality comprises one or more of an insertion, deletion or duplication. In some instances, the insertion, deletion or duplication is at least 1%, 2%, 3%, 4%, 5%, 6%, 7%, 8%, 9%, 10%, 12%, 15%, or at least 20% of the total chromosome length. In some instances, the insertion, deletion or duplication is about 1%, 2%, 3%, 4%, 5%, 6%, 7%, 8%, 9%, 10%, 12%, 15%, or about 20% of the total chromosome length. In some instances, the insertion, deletion or duplication is no more than 1%, 2%, 3%, 4%, 5%, 6%, 7%, 8%, 9%, 10%, 12%, 15%, or no more than 20% of the total chromosome length. In some instances, the insertion, deletion or duplication is 1%-30%, 1%-20%, 1%-15%, 1%-10%, 1%-5%, 2%-20%, 3%-25%, 4%-20%, 5%-15%, 5%-30%, 5%-20%, 10%-30%, or 15%-30% of the total chromosome length.

In some instances, the methods (e.g., PTA) described herein result in higher detection sensitivity and/or lower rates of false positives for the detection of fetal genetic abnormalities. In some instances a mutation is a difference between an analyzed sequence (e.g., using the methods described herein) and a reference sequence. Reference sequences are in some instances obtained from other organisms, other individuals of the same or similar species, other cells in the same organism, populations of organisms, or other areas of the same genome. In some instances, mutations are identified on a plasmid or chromosome. In some instances, a mutation is an SNV (single nucleotide variation), SNP (single nucleotide polymorphism), or CNV (copy number variation, or CNA/copy number aberration). In some instances, a mutation is base substitution, insertion, or deletion. In some instances, a mutation is a transition, transversion, nonsense mutation, silent mutation, synonymous or non-synonymous mutation, non-pathogenic mutation, missense mutation, or frameshift mutation (deletion or insertion). In some instances, PTA results in higher detection sensitivity and/or lower rates of false positives for the detection of mutations when compared to methods such as in-silico prediction, ChIP-seq, GUIDE-seq, circle-seq, HTGTS (High-Throughput Genome-Wide Translocation Sequencing), IDLV (integration-deficient lentivirus), Digenome-seq, FISH (fluorescence in situ hybridization), or DISCOVER-seq. In some instances, a fetal genetic abnormality is detected with at sensitivity of at least 0.001%, 0.01%, 0.1%, 0.5%, 1%, 2%, 5%, 10%, or at least 20%.

Genetic abnormalities may be linked to specific genetic diseases. In some instances, methods described herein such as PTA are used to identify genetic diseases. In some instances, the disease is caused by a chromosomal abnormality. In some instances, the disease comprises Down syndrome, Patau syndrome, Klinefelter Syndrome, Turner Syndrome, or Edwards Syndrome. In some instances, the disease is caused by a single gene defect. In some instances, the disease comprises phenylketonuria (PKU), sickle-cell anemia, Beta Thalassemia, Tay-Sachs disease, Sandhoff disease, or cystic fibrosis (CF). In some instances the disease comprises achondroplasia, congenital adrenal hyperplasia, Cystic fibrosis, Down syndrome, fragile XD syndrome, Hemophilia A, Huntington's disease, Muscular dystrophy, Polycystic kidney disease, Sickle cell disease, Tay-Sachs disease, trisomy 21, trisomy 18, trisomy 13, Turner syndrome, spina bifida, anencephaly, or Thalassemia.

Described herein are methods, devices, and compositions for high-throughput analysis of single cells. Analysis of cells in bulk provides general information about the cell population, but often is unable to detect low-frequency mutants over the background. Such mutants may comprise important properties such as drug resistance or mutations associated with cancer. In some instances, DNA, RNA, and/or proteins from the same single cell are analyzed in parallel, using the devices described herein. The analysis may include identification of epigenetic post-translational (e.g., glycosylation, phosphorylation, acetylation, ubiquitination, histone modification) and/or post-transcriptional (e.g., methylation, hydroxymethylation) modifications. Such methods may comprise “Primary Template-Directed Amplification” (PTA) to obtain libraries of nucleic acids for sequencing. In some instances PTA is combined with additional steps or methods such as RT-PCR or proteome/protein quantification techniques (e.g., mass spectrometry, antibody staining, etc.). In some instances, various components of a cell are physically or spatially separated from each other during individual analysis steps. In some instances, proteins are first labeled with antibodies. In some instances, at least some of the antibodies comprise a tag or marker (e.g., nucleic acid/oligo tag, mass tag, or fluorescent, tag). In some instances, a portion of the antibodies comprise an oligo tag. In some instances, a portion of the antibodies comprise a fluorescent marker. In some instances antibodies are labeled by two or more tags or markers. In some instances, a portion of the antibodies are sorted based on fluorescent markers. After RT-PCR, first strand mRNA products are generated and then removed for analysis. Libraries are then generated from RT-PCR products and barcodes present on protein-specific antibodies, which are subsequently sequenced. In parallel, genomic DNA from the same cell is subjected to PTA, a library generated, and sequenced. Sequencing results from the genome, proteome, and transcriptome are in some instances pooled using bioinformatics methods. Methods described herein in some instances comprise any combination of labeling, cell sorting, affinity separation/purification, lysing of specific cell components (e.g., outer membrane, nucleus, etc.), RNA amplification, DNA amplification (e.g., PTA), or other step associated with protein, RNA, or DNA isolation or analysis. In some instances, methods described herein comprise one or more enrichment steps, such as exome enrichment.

### Sample Preparation and Isolation of Single Cells

Methods described herein may require isolation of single cells for analysis. Any method of single cell isolation may be used with PTA, such as mouth pipetting, micro pipetting, flow cytometry/FACS, microfluidics, methods of sorting nuclei (tetraploid or other), or manual dilution. Such methods are aided by additional reagents and steps, for example, antibody-based enrichment (e.g., circulating tumor cells), other small-molecule or protein-based enrichment methods, or fluorescent labeling. In some instances, a method of multiomic analysis described herein comprises mechanical or enzymatic dissociate of cells from larger tissues. In some instances, cells are isolated using a robotic device comprising a capillary. In some instances, embryonic cells are isolated from a pre-implantation embryo or endocervical sample. In some instances, multiomic methods are utilized to analyze nucleic acid and protein analytes from a sample, such as fetal cells.

### Sample Preparation of Multiple Cells

Methods described herein may comprise obtaining a plurality of embryonic cells. In some embodiments, a plurality of embryonic cells are pooled. In some embodiments, the methods comprise isolating nucleic acids using the methods described herein from a plurality of embryonic cells.

In some embodiments, the methods comprise obtaining at least 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 105, 110, 120, 130, 140, 150, 160, 170, 180, 190, 200, 210, 220, 230, 240, 250, 260, 270, 280, 290, 300, or more than 300 cells. In some embodiments, the methods comprise obtaining from between about 1 and 200 cells, 2 and 200 cells, 3 and 200 cells, 4 and 200 cells, 5 and 200 cells, 10 and 200 cells, 20 and 200 cells, 30 and 200 cells, 40 and 200 cells, 50 and 200 cells, 60 and 200 cells, 70 and 200 cells, 80 and 200 cells, 90 and 200 cells, 100 and 200 cells.

In some embodiments, the methods comprise isolating nucleic acids from at least 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 105, 110, 120, 130, 140, 150, 160, 170, 180, 190, 200, 210, 220, 230, 240, 250, 260, 270, 280, 290, 300, or more than 300 cells. In some embodiments, the methods comprise isolating nucleic acids from between about 1 and 200 cells, 2 and 200 cells, 3 and 200 cells, 4 and 200 cells, 5 and 200 cells, 10 and 200 cells, 20 and 200 cells, 30 and 200 cells, 40 and 200 cells, 50 and 200 cells, 60 and 200 cells, 70 and 200 cells, 80 and 200 cells, 90 and 200 cells, 100 and 200 cells.

### Preparation and Analysis of Cell Components

Methods of multiomic analysis comprising PTA described herein may comprise one or more methods of processing cell components such as DNA, RNA, and/or proteins. In some instances, the nucleus (comprising genomic DNA) is physically separated from the cytosol (comprising mRNA), followed by a membrane-selective lysis buffer to dissolve the membrane but keep the nucleus intact. The cytosol is then separated from the nucleus using methods including micro pipetting, centrifugation, or anti-body conjugated magnetic microbeads. In another instance, an oligo-dT primer coated magnetic bead binds polyadenylated mRNA for separation from DNA. In another instance, DNA and RNA are preamplified simultaneously, and then separated for analysis. In another instance, a single cell is split into two equal pieces, with mRNA from one half processed, and genomic DNA from the other half processed. In some instances, methods described herein are conducted on genomic DNA, RNA, or both genomic DNA and RNA.

Methods described herein (e.g., PTA) may be used as a replacement for any number of other known methods in the art which are used for single cell sequencing (multiomics or the like). PTA may substitute genomic DNA sequencing methods such as MDA, PicoPlex, DOP-PCR, MALBAC, or target-specific amplifications. In some instances, PTA replaces the standard genomic DNA sequencing method in a multiomics method including DR-seq (Dey et al., 2015), G&T seq (MacAulay et al., 2015), scMT-seq (Hu et al., 2016), sc-GEM (Cheow et al., 2016), sc Trio-seq (Hou et al., 2016), simultaneous multiplexed measurement of RNA and proteins (Darmanis et al., 2016), scCOOL-seq (Guo et al., 2017), CITE-seq (Stoeckius et al., 2017), REAP-seq (Peterson et al., 2017), scNMT-seq (Clark et al., 2018), or SIDR-seq (Han et al., 2018). In some instances, a method described herein comprises PTA and a method of polyadenylated mRNA transcripts. In some instances, a method described herein comprises PTA and a method of non-polyadenylated mRNA transcripts. In some instances, a method described herein comprises PTA and a method of total (polyadenylated and non-polyadenylated) mRNA transcripts.

In some instances, PTA is combined with a standard RNA sequencing method to obtain genome and transcriptome data. In some instances, a multiomics method described herein comprises PTA and one of the following: Drop-seq (Macosko, et al. 2015), mRNA-seq (Tang et al., 2009), InDrop (Klein et al., 2015), MARS-seq (Jaitin et al., 2014), Smart-seq2 (Hashimshony, et al., 2012; Fish et al., 2016), CEL-seq (Jaitin et al., 2014), STRT-seq (Islam, et al., 2011), Quartz-seq (Sasagawa et al., 2013), CEL-seq2 (Hashimshony, et al. 2016), cytoSeq (Fan et al., 2015), SuPeR-seq (Fan et al., 2011), RamDA-seq (Hayashi, et al. 2018), MATQ-seq (Sheng et al., 2017), or SMARTer (Verboom et al., 2019).

Various reaction conditions and mixes may be used for generating cDNA libraries for transcriptome analysis. In some instances, an RT reaction mix is used to generate a cDNA library. In some instances, the RT reaction mixture comprises a crowding reagent, at least one primer, a template switching oligonucleotide (TSO), a reverse transcriptase, and a dNTP mix. In some instances, an RT reaction mix comprises an RNAse inhibitor. In some instances an RT reaction mix comprises one or more surfactants. In some instances an RT reaction mix comprises Tween-20 and/or Triton-X. In some instances an RT reaction mix comprises Betaine. In some instances an RT reaction mix comprises one or more salts. In some instances an RT reaction mix comprises a magnesium salt (e.g., magnesium chloride) and/or tetramethylammonium chloride. In some instances an RT reaction mix comprises gelatin. In some instances an RT reaction mix comprises PEG (PEG1000, PEG2000, PEG4000, PEG6000, PEG8000, or PEG of other length).

Multiomic methods described herein may provide both genomic and RNA transcript information from a single cell (e.g., a combined or dual protocol). In some instances, genomic information from the single cell is obtained from the PTA method, and RNA transcript information is obtained from reverse transcription to generate a cDNA library. In some instances, a whole transcript method is used to obtain the cDNA library. In some instances, 3′ or 5′ end counting is used to obtain the cDNA library. In some instances, cDNA libraries are not obtained using UMIs. In some instances, a multiomic method provides RNA transcript information from the single cell for at least 500, 1000, 2000, 5000, 8000, 10,000, 12,000, or at least 15,000 genes. In some instances, a multiomic method provides RNA transcript information from the single cell for about 500, 1000, 2000, 5000, 8000, 10,000, 12,000, or about 15,000 genes. In some instances, a multiomic method provides RNA transcript information from the single cell for 100-12,000 1000-10,000, 2000-15,000, 5000-15,000, 10,000-20,000, 8000-15,000, or 10,000-15,000 genes. In some instances, a multiomic method provides genomic sequence information for at least 80%, 90%, 92%, 95%, 97%, 98%, or at least 99% of the genome of the single cell. In some instances, a multiomic method provides genomic sequence information for about 80%, 90%, 92%, 95%, 97%, 98%, or about 99% of the genome of the single cell.

Multiomic methods may comprise analysis of single cells from a population of cells. In some instances, at least 5, 10, 20, 50, 100, 200, 500, 1000, 2000, 5000, or at least 8000 cells are analyzed. In some instances, about 5, 10, 20, 50, 100, 200, 500, 1000, 2000, 5000, or about 8000 cells are analyzed. In some instances, 5-100, 10-100, 50-500, 100-500, 100-1000, 50-5000, 100-5000, 500-1000, 500-10000, 1000-10000, or 5000-20,000 cells are analyzed.

Multiomic methods may generate yields of genomic DNA from the PTA reaction based on the type of single cell. In some instances, the amount of DNA generated from a single cell is about 0.1, 1, 1.5, 2, 3, 5, or about 10 micrograms. In some instances, the amount of DNA generated from a single cell is about 0.1, 1, 1.5, 2, 3, 5, or about 10 femtograms. In some instances, the amount of DNA generated from a single cell is at least 0.1, 1, 1.5, 2, 3, 5, or at least 10 micrograms. In some instances, the amount of DNA generated from a single cell is at least 0.1, 1, 1.5, 2, 3, 5, or at least 10 femtograms. In some instances, the amount of DNA generated from a single cell is about 0.1-10, 1-10, 1.5-10, 2-20, 2-50, 1-3, or 0.5-3.5 micrograms. In some instances, the amount of DNA generated from a single cell is about 0.1-10, 1-10, 1.5-10, 2-20, 2-4, 1-3, or 0.5-4 femtograms.

### Methylome Analysis

Described herein are methods comprising PTA, wherein sites of methylated DNA in single cells are determined using the PTA method. In some instances, sites of methylated DNA are detected using enzymatic methods. In some instances, sites of methylated DNA are detected using non-enzymatic methods. In some instances, these methods further comprise parallel analysis of the transcriptome and/or proteome of the same cell. Methods of detecting methylated genomic bases include selective restriction with methylation-sensitive endonucleases, followed by processing with the PTA method. Sites cut by such enzymes are determined from sequencing, and methylated bases are identified. In some instance, libraries are amplified with methylation-specific primers which selectively anneal to methylated sequences.

In another instance, bisulfite treatment of genomic DNA libraries is used to detect a methylation signature. Bisulfite conversion of DNA results in conversion of unmodified cytosine (C) to uracil (U) that will be read as thymine (T) upon sequencing of PCR amplified DNA. Both 5meC and 5hmC are protected against conversion and will not be converted to U. Therefore they will both be read as C upon sequencing. Alternatively, non-methylation-specific PCR is conducted, followed by one or more methods to discriminate between bisulfite-reacted bases, including direct pyrosequencing, MS-SnuPE, HRM, COBRA, MS-SSCA, or base-specific cleavage/MALDI-TOF. In some instances, genomic DNA samples are split for parallel analysis of the genome (or an enriched portion thereof) and methylome analysis. In some instances, analysis of the genome and methylome comprises enrichment of genomic fragments (e.g., exome, or other targets) or whole genome sequencing.

In some instances, the methylation signature is preserved during PTA. In some instances, processing with the PTA method while preserving the methylation signature is used to create a reference library. In some instances, after a reference library is created, methylation patterns are detected using the methods described herein to create a methylation-specific library. In some embodiments, the methylation-specific library is compared to the reference library. In some instances, the methylation-specific library and the reference library are prepared from the same cell. In some instances, comparing the methylation-specific library to the reference library allows for identification of a methylation signature. In some instances, after a reference library is created, the genomic DNA library is treated with bisulfite. In some instances, the genomic library treated with bisulfite is amplified with the PTA method to produce a methylation-specific library.

### Bioinformatics

The data obtained from single-cell analysis methods utilizing PTA described herein may be compiled into a database. Described herein are methods and systems of bioinformatic data integration. Data from the proteome, genome, transcriptome, methylome or other data is in some instances combined/integrated into a database and analyzed. Bioinformatic data integration methods and systems in some instances comprise one or more of protein detection (FACS and/or NGS), mRNA detection, and/or genome variance detection. In some instances, this data is correlated with a disease state or condition. In some instances, data from a plurality of single cells is compiled to describe properties of a larger cell population, such as cells from a specific sample, region, organism, or tissue. In some instances, protein data is acquired from fluorescently labeled antibodies which selectively bind to proteins on a cell. In some instances, a method of protein detection comprises grouping cells based on fluorescent markers and reporting sample location post-sorting. In some instances, a method of protein detection comprises detecting sample barcodes, detecting protein barcodes, comparing to designed sequences, and grouping cells based on barcode and copy number. In some instances, protein data is acquired from barcoded antibodies which selectively bind to proteins on a cell. In some instances, transcriptome data is acquired from sample and RNA specific barcodes. In some instances, a method of mRNA detection comprises detecting sample and RNA specific barcodes, aligning to genome, aligning to RefSeq/Encode, reporting Exon/Intro/Intergenic sequences, analyzing exon-exon junctions, grouping cells based on barcode and expression variance and clustering analysis of variance and top variable genes. In some instances, genomic data is acquired from sample and DNA specific barcodes. In some instances, a method of genome variance detection comprises detecting sample and DNA specific barcodes, aligning to the genome, determine genome recovery and SNV mapping rate, filtering reads on exon-exon junctions, generating variant call file (VCF), and clustering analysis of variance and top variable mutations. In some instances in-silico bioinformatic methods are used to filter one or more nucleic acid sequences from sequencing data. In some instances, maternal nucleic acid sequences are filtered from sequencing data comprising both fetal and maternal nucleic acid sequences.

### Primary Template-Directed Amplification

Described herein are nucleic acid amplification methods, such as “Primary Template-Directed Amplification (PTA).” In some instances, PTA is combined with other analysis workflows for multiomic analysis. With the PTA method, amplicons are preferentially generated from the primary template (“direct copies”) using a polymerase (e.g., a strand displacing polymerase). Consequently, errors are propagated at a lower rate from daughter amplicons during subsequent amplifications compared to MDA. The result is an easily executed method that, unlike existing WGA protocols, can amplify low DNA input including the genomes of single cells with high coverage breadth and uniformity in an accurate and reproducible manner. In some instances, PTA enables kinetic control of an amplification reaction. In some instances, PTA results in a pseudo-linear amplification reaction (rather than exponential amplification). Moreover, the terminated amplification products can undergo direction ligation after removal of the terminators, allowing for the attachment of a cell barcode to the amplification primers so that products from all cells can be pooled after undergoing parallel amplification reactions. In some instances, template nucleic acids are not bound to a solid support. In some instances, direct copies of template nucleic acids are not bound to a solid support. In some instances, one or more primers are not bound to a solid support. In some instances, no primers are not bound to a solid support. In some instances, a primer is attached to a first solid support, and a template nucleic acid is attached to a second solid support, wherein the first and the second solid supports are not the same. In some instances, PTA is used to analyze single cells from a larger population of cells. In some instances, PTA is used to analyze more than one cell from a larger population of cells, or an entire population of cells.

Described herein are methods employing nucleic acid polymerases with strand displacement activity for amplification. In some instances, such polymerases comprise strand displacement activity and low error rate. In some instances, such polymerases comprise strand displacement activity and proofreading exonuclease activity, such as 3′->5′ proofreading activity. In some instances, nucleic acid polymerases are used in conjunction with other components such as reversible or irreversible terminators, or additional strand displacement factors. In some instances, the polymerase has strand displacement activity, but does not have exonuclease proofreading activity. For example, in some instances such polymerases include bacteriophage phi29 (Φ29) polymerase, which also has very low error rate that is the result of the 3′->5′ proofreading exonuclease activity (see, e.g., U.S. Pat. Nos. 5,198,543 and 5,001,050). In some instances, non-limiting examples of strand displacing nucleic acid polymerases include, e.g., genetically modified phi29 (Ø29) DNA polymerase, Klenow Fragment of DNA polymerase I (Jacobsen et al., Eur. J. Biochem. 45:623-627 (1974)), phage M2 DNA polymerase (Matsumoto et al., Gene 84:247 (1989)), phage phiPRD1 DNA polymerase (Jung et al., Proc. Natl. Acad. Sci. USA 84:8287 (1987); Zhu and Ito, Biochim. Biophys. Acta. 1219:267-276 (1994)), Bst DNA polymerase (e.g., Bst large fragment DNA polymerase (Exo(−) Bst; Aliotta et al., Genet. Anal. (Netherlands) 12:185-195 (1996)), exo(−)Bca DNA polymerase (Walker and Linn, Clinical Chemistry 42:1604-1608 (1996)), Bsu DNA polymerase, Vent_{R} DNA polymerase including Vent_{R}(exo−) DNA polymerase (Kong et al., J. Biol. Chem. 268:1965-1975 (1993)), Deep Vent DNA polymerase including Deep Vent (exo−) DNA polymerase, IsoPol DNA polymerase, DNA polymerase I, Therminator DNA polymerase, T5 DNA polymerase (Chatterjee et al., Gene 97:13-19 (1991)), Sequenase (U.S. Biochemicals), T7 DNA polymerase, T7-Sequenase, T7 gp5 DNA polymerase, PRDI DNA polymerase, T4 DNA polymerase (Kaboord and Benkovic, Curr. Biol. 5:149-157 (1995)). Additional strand displacing nucleic acid polymerases are also compatible with the methods described herein. The ability of a given polymerase to carry out strand displacement replication can be determined, for example, by using the polymerase in a strand displacement replication assay (e.g., as disclosed in U.S. Pat. No. 6,977,148). Such assays in some instances are performed at a temperature suitable for optimal activity for the enzyme being used, for example, 32° C. for phi29 DNA polymerase, from 46° C. to 64° C. for exo(−) Bst DNA polymerase, or from about 60° C. to 70° C. for an enzyme from a hyperthermophylic organism. Another useful assay for selecting a polymerase is the primer-block assay described in Kong et al., J. Biol. Chem. 268:1965-1975 (1993). The assay consists of a primer extension assay using an M13 ssDNA template in the presence or absence of an oligonucleotide that is hybridized upstream of the extending primer to block its progress. Other enzymes capable of displacement the blocking primer in this assay are in some instances useful for the disclosed method. In some instances, polymerases incorporate dNTPs and terminators at approximately equal rates. In some instances, the ratio of rates of incorporation for dNTPs and terminators for a polymerase described herein are about 1:1, about 1.5:1, about 2:1, about 3:1 about 4:1 about 5:1, about 10:1, about 20:1 about 50:1, about 100:1, about 200:1, about 500:1, or about 1000:1. In some instances, the ratio of rates of incorporation for dNTPs and terminators for a polymerase described herein are 1:1 to 1000:1, 2:1 to 500:1, 5:1 to 100:1, 10:1 to 1000:1, 100:1 to 1000:1, 500:1 to 2000:1, 50:1 to 1500:1, or 25:1 to 1000:1.

Described herein are methods of amplification wherein strand displacement can be facilitated through the use of a strand displacement factor, such as, e.g., helicase. Such factors are in some instances used in conjunction with additional amplification components, such as polymerases, terminators, or other component. In some instances, a strand displacement factor is used with a polymerase that does not have strand displacement activity. In some instances, a strand displacement factor is used with a polymerase having strand displacement activity. Without being bound by theory, strand displacement factors may increase the rate that smaller, double stranded amplicons are reprimed. In some instances, any DNA polymerase that can perform strand displacement replication in the presence of a strand displacement factor is suitable for use in the PTA method, even if the DNA polymerase does not perform strand displacement replication in the absence of such a factor. Strand displacement factors useful in strand displacement replication in some instances include (but are not limited to) BMRF1 polymerase accessory subunit (Tsurumi et al., J. Virology 67 (12): 7648-7653 (1993)), adenovirus DNA-binding protein (Zijderveld and van der Vliet, J. Virology 68 (2): 1158-1164 (1994)), herpes simplex viral protein ICP8 (Boehmer and Lehman, J. Virology 67 (2): 711-715 (1993); Skaliter and Lehman, Proc. Natl. Acad. Sci. USA 91 (22): 10665-10669 (1994)); single-stranded DNA binding proteins (SSB; Rigler and Romano, J. Biol. Chem. 270:8910-8919 (1995)); phage T4 gene 32 protein (Villemain and Giedroc, Biochemistry 35:14395-14404 (1996); T7 helicase-primase; T7 gp2.5 SSB protein; Tte-UvrD (from Thermoanaerobacter tengcongensis), calf thymus helicase (Siegel et al., J. Biol. Chem. 267:13629-13635 (1992)); bacterial SSB (e.g., E. coli SSB), Replication Protein A (RPA) in eukaryotes, human mitochondrial SSB (mtSSB), and recombinases, (e.g., Recombinase A (RecA) family proteins, T4 UvsX, T4 UvsY, Sak4 of Phage HK620, Rad51, Dmc1, or Radb). Combinations of factors that facilitate strand displacement and priming are also consistent with the methods described herein. For example, a helicase is used in conjunction with a polymerase. In some instances, the PTA method comprises use of a single-strand DNA binding protein (SSB, T4 gp32, or other single stranded DNA binding protein), a helicase, and a polymerase (e.g., SauDNA polymerase, Bsu polymerase, Bst2.0, GspM, GspM2.0, GspSSD, or other suitable polymerase). In some instances, reverse transcriptases are used in conjunction with the strand displacement factors described herein. In some instances, reverse transcriptases are used in conjunction with the strand displacement factors described herein. In some instances, amplification is conducted using a polymerase and a nicking enzyme (e.g., “NEAR”), such as those described in U.S. Pat. No. 9,617,586. In some instances, the nicking enzyme is Nt. BspQI, Nb.BbvCi, Nb.BsmI, Nb.BsrDI, Nb.BtsI, Nt.AlwI, Nt. BbvCI, Nt.BstNBI, Nt.CviPII, Nb.Bpu10I, or Nt. Bpu10I.

Described herein are amplification methods comprising use of terminator nucleotides, polymerases, and additional factors or conditions. For example, such factors are used in some instances to fragment the nucleic acid template(s) or amplicons during amplification. In some instances, such factors comprise endonucleases. In some instances, factors comprise transposases. In some instances, mechanical shearing is used to fragment nucleic acids during amplification. In some instances, nucleotides are added during amplification that may be fragmented through the addition of additional proteins or conditions. For example, uracil is incorporated into amplicons; treatment with uracil D-glycosylase fragments nucleic acids at uracil-containing positions. Additional systems for selective nucleic acid fragmentation are also in some instances employed, for example an engineered DNA glycosylase that cleaves modified cytosine-pyrene base pairs. (Kwon, et al. Chem Biol. 2003, 10(4), 351)

Described herein are amplification methods comprising use of terminator nucleotides, which terminate nucleic acid replication thus decreasing the size of the amplification products. Such terminators are in some instances used in conjunction with polymerases, strand displacement factors, or other amplification components described herein. In some instances, terminator nucleotides reduce or lower the efficiency of nucleic acid replication. Such terminators in some instances reduce extension rates by at least 99.9%, 99%, 98%, 95%, 90%, 85%, 80%, 75%, 70%, or at least 65%. Such terminators in some instances reduce extension rates by 50%-90%, 60%-80%, 65%-90%, 70%-85%, 60%-90%, 70%-99%, 80%-99%, or 50%-80%. In some instances terminators reduce the average amplicon product length by at least 99.9%, 99%, 98%, 95%, 90%, 85%, 80%, 75%, 70%, or at least 65%. Terminators in some instances reduce the average amplicon length by 50%-90%, 60%-80%, 65%-90%, 70%-85%, 60%-90%, 70%-99%, 80%-99%, or 50%-80%. In some instances, amplicons comprising terminator nucleotides form loops or hairpins which reduce a polymerase's ability to use such amplicons as templates. Use of terminators in some instances slows the rate of amplification at initial amplification sites through the incorporation of terminator nucleotides (e.g., dideoxynucleotides that have been modified to make them exonuclease-resistant to terminate DNA extension), resulting in smaller amplification products. By producing smaller amplification products than the currently used methods (e.g., average length of 50-2000 nucleotides in length for PTA methods as compared to an average product length of >10,000 nucleotides for MDA methods) PTA amplification products in some instances undergo direct ligation of adapters without the need for fragmentation, allowing for efficient incorporation of cell barcodes and unique molecular identifiers (UMI).

Terminator nucleotides are present at various concentrations depending on factors such as polymerase, template, or other factors. For example, the amount of terminator nucleotides in some instances is expressed as a ratio of non-terminator nucleotides to terminator nucleotides in a method described herein. Such concentrations in some instances allow control of amplicon lengths. In some instances, the ratio of terminator to non-terminator nucleotides is modified for the amount of template present or the size of the template. In some instances, the ratio of ratio of terminator to non-terminator nucleotides is reduced for smaller samples sizes (e.g., femtogram to picogram range). In some instances, the ratio of non-terminator to terminator nucleotides is about 2:1, 5:1, 7:1, 10:1, 20:1, 50:1, 100:1, 200:1, 500:1, 1000:1, 2000:1, or 5000:1. In some instances the ratio of non-terminator to terminator nucleotides is 2:1-10:1, 5:1-20:1, 10:1-100:1, 20:1-200:1, 50:1-1000:1, 50:1-500:1, 75:1-150:1, or 100:1-500:1. In some instances, at least one of the nucleotides present during amplification using a method described herein is a terminator nucleotide. Each terminator need not be present at approximately the same concentration; in some instances, ratios of each terminator present in a method described herein are optimized for a particular set of reaction conditions, sample type, or polymerase. Without being bound by theory, each terminator may possess a different efficiency for incorporation into the growing polynucleotide chain of an amplicon, in response to pairing with the corresponding nucleotide on the template strand. For example, in some instances a terminator pairing with cytosine is present at about 3%, 5%, 10%, 15%, 20%, 25%, or 50% higher concentration than the average terminator concentration. In some instances a terminator pairing with thymine is present at about 3%, 5%, 10%, 15%, 20%, 25%, or 50% higher concentration than the average terminator concentration. In some instances a terminator pairing with guanine is present at about 3%, 5%, 10%, 15%, 20%, 25%, or 50% higher concentration than the average terminator concentration. In some instances a terminator pairing with adenine is present at about 3%, 5%, 10%, 15%, 20%, 25%, or 50% higher concentration than the average terminator concentration. In some instances a terminator pairing with uracil is present at about 3%, 5%, 10%, 15%, 20%, 25%, or 50% higher concentration than the average terminator concentration. Any nucleotide capable of terminating nucleic acid extension by a nucleic acid polymerase in some instances is used as a terminator nucleotide in the methods described herein. In some instances, a reversible terminator is used to terminate nucleic acid replication. In some instances, a non-reversible terminator is used to terminate nucleic acid replication. In some instances, non-limited examples of terminators include reversible and non-reversible nucleic acids and nucleic acid analogs, such as, e.g., 3′ blocked reversible terminator comprising nucleotides, 3′ unblocked reversible terminator comprising nucleotides, terminators comprising 2′ modifications of deoxynucleotides, terminators comprising modifications to the nitrogenous base of deoxynucleotides, or any combination thereof. In one embodiment, terminator nucleotides are dideoxynucleotides. Other nucleotide modifications that terminate nucleic acid replication and may be suitable for practicing the invention include, without limitation, any modifications of the r group of the 3′ carbon of the deoxyribose such as inverted dideoxynucleotides, 3′ biotinylated nucleotides, 3′ amino nucleotides, 3′-phosphorylated nucleotides, 3′-O-methyl nucleotides, 3′ carbon spacer nucleotides including 3′ C3 spacer nucleotides, 3′ C18 nucleotides, 3′ Hexanediol spacer nucleotides, acyclonucleotides, and combinations thereof. In some instances, terminators are polynucleotides comprising 1, 2, 3, 4, or more bases in length. In some instances, terminators do not comprise a detectable moiety or tag (e.g., mass tag, fluorescent tag, dye, radioactive atom, or other detectable moiety). In some instances, terminators do not comprise a chemical moiety allowing for attachment of a detectable moiety or tag (e.g., “click” azide/alkyne, conjugate addition partner, or other chemical handle for attachment of a tag). In some instances, all terminator nucleotides comprise the same modification that reduces amplification to at region (e.g., the sugar moiety, base moiety, or phosphate moiety) of the nucleotide. In some instances, at least one terminator has a different modification that reduces amplification. In some instances, all terminators have a substantially similar fluorescent excitation or emission wavelengths. In some instances, terminators without modification to the phosphate group are used with polymerases that do not have exonuclease proofreading activity. Terminators, when used with polymerases which have 3′->5′ proofreading exonuclease activity (such as, e.g., phi29) that can remove the terminator nucleotide, are in some instances further modified to make them exonuclease-resistant. For example, dideoxynucleotides are modified with an alpha-thio group that creates a phosphorothioate linkage which makes these nucleotides resistant to the 3′->5′ proofreading exonuclease activity of nucleic acid polymerases. Such modifications in some instances reduce the exonuclease proofreading activity of polymerases by at least 99.5%, 99%, 98%, 95%, 90%, or at least 85%. Non-limiting examples of other terminator nucleotide modifications providing resistance to the 3′->5′ exonuclease activity include in some instances: nucleotides with modification to the alpha group, such as alpha-thio dideoxynucleotides creating a phosphorothioate bond, C3 spacer nucleotides, locked nucleic acids (LNA), inverted nucleic acids, 2′ Fluoro bases, 3′ phosphorylation, 2′-O-Methyl modifications (or other 2′-O-alkyl modification), propyne-modified bases (e.g., deoxycytosine, deoxyuridine), L-DNA nucleotides, L-RNA nucleotides, nucleotides with inverted linkages (e.g., 5′-5′ or 3′-3′), 5′ inverted bases (e.g., 5′ inverted 2′,3′-dideoxy dT), methylphosphonate backbones, and trans nucleic acids. In some instances, nucleotides with modification include base-modified nucleic acids comprising free 3′ OH groups (e.g., 2-nitrobenzyl alkylated HOMedU triphosphates, bases comprising modification with large chemical groups, such as solid supports or other large moiety). In some instances, a polymerase with strand displacement activity but without 3′->5′exonuclease proofreading activity is used with terminator nucleotides with or without modifications to make them exonuclease resistant. Such nucleic acid polymerases include, without limitation, Bst DNA polymerase, Bsu DNA polymerase, Deep Vent (exo−) DNA polymerase, Klenow Fragment (exo−) DNA polymerase, Therminator DNA polymerase, and Vent_{R} (exo−).

### Primers and Amplicon Libraries

Described herein are amplicon libraries resulting from amplification of at least one target nucleic acid molecule. Such libraries are in some instances generated using the methods described herein, such as those using terminators. Such methods comprise use of strand displacement polymerases or factors, terminator nucleotides (reversible or irreversible), or other features and embodiments described herein. In some instances, reversible terminators are capable of removal by an exonuclease (e.g., or polymerase having exonuclease activity). In some instances, irreversible terminators are not capable of substantial removal by an exonuclease (e.g., or polymerase having exonuclease activity). In some instances, amplicon libraries generated by use of terminators described herein are further amplified in a subsequent amplification reaction (e.g., PCR). In some instances, subsequent amplification reactions do not comprise terminators. In some instances, amplicon libraries comprise polynucleotides, wherein at least 50%, 60%, 70%, 80%, 90%, 95%, or at least 98% of the polynucleotides comprise at least one terminator nucleotide. In some instances, the amplicon library comprises the target nucleic acid molecule from which the amplicon library was derived. The amplicon library comprises a plurality of polynucleotides, wherein at least some of the polynucleotides are direct copies (e.g., replicated directly from a target nucleic acid molecule, such as genomic DNA, RNA, or other target nucleic acid). For example, at least 5%, 10%, 20%, 30%, 40%, 50%, 60%, 70%, 80%, 90%, 95% or more than 95% of the amplicon polynucleotides are direct copies of the at least one target nucleic acid molecule. In some instances, at least 5% of the amplicon polynucleotides are direct copies of the at least one target nucleic acid molecule. In some instances, at least 10% of the amplicon polynucleotides are direct copies of the at least one target nucleic acid molecule. In some instances, at least 15% of the amplicon polynucleotides are direct copies of the at least one target nucleic acid molecule. In some instances, at least 20% of the amplicon polynucleotides are direct copies of the at least one target nucleic acid molecule. In some instances, at least 50% of the amplicon polynucleotides are direct copies of the at least one target nucleic acid molecule. In some instances, 3%-5%, 3-10%, 5%-10%, 10%-20%, 20%-30%, 30%-40%, 5%-30%, 10%-50%, or 15%-75% of the amplicon polynucleotides are direct copies of the at least one target nucleic acid molecule. In some instances, at least some of the polynucleotides are direct copies of the target nucleic acid molecule, or daughter (a first copy of the target nucleic acid) progeny. For example, at least 5%, 10%, 20%, 30%, 40%, 50%, 60%, 70%, 80%, 90%, 95% or more than 95% of the amplicon polynucleotides are direct copies of the at least one target nucleic acid molecule or daughter progeny. In some instances, at least 5% of the amplicon polynucleotides are direct copies of the at least one target nucleic acid molecule or daughter progeny. In some instances, at least 10% of the amplicon polynucleotides are direct copies of the at least one target nucleic acid molecule or daughter progeny. In some instances, at least 20% of the amplicon polynucleotides are direct copies of the at least one target nucleic acid molecule or daughter progeny. In some instances, at least 30% of the amplicon polynucleotides are direct copies of the at least one target nucleic acid molecule or daughter progeny. In some instances, 3%-5%, 3%-10%, 5%-10%, 10%-20%, 20%-30%, 30%-40%, 5%-30%, 10%-50%, or 15%-75% of the amplicon polynucleotides are direct copies of the at least one target nucleic acid molecule or daughter progeny. In some instances, direct copies of the target nucleic acid are 50-2500, 75-2000, 50-2000, 25-1000, 50-1000, 500-2000, or 50-2000 bases in length. In some instances, daughter progeny are 1000-5000, 2000-5000, 1000-10,000, 2000-5000, 1500-5000, 3000-7000, or 2000-7000 bases in length. In some instances, the average length of PTA amplification products is 25-3000 nucleotides in length, 50-2500, 75-2000, 50-2000, 25-1000, 50-1000, 500-2000, or 50-2000 bases in length. In some instance, amplicons generated from PTA are no more than 5000, 4000, 3000, 2000, 1700, 1500, 1200, 1000, 700, 500, or no more than 300 bases in length. In some instance, amplicons generated from PTA are 1000-5000, 1000-3000, 200-2000, 200-4000, 500-2000, 750-2500, or 1000-2000 bases in length. Amplicon libraries generated using the methods described herein in some instances comprise at least 1000, 2000, 5000, 10,000, 100,000, 200,000, 500,000 or more than 500,000 amplicons comprising unique sequences. In some instances, the library comprises at least 100, 200, 300, 400, 500, 600, 700, 800, 900, 1000, 1100, 1200, 1300, 1400, 1500, 2000, 2500, 3000, or at least 3500 amplicons. In some instances, at least 5%, 10%, 15%, 20%, 25%, 30% or more than 30% of amplicon polynucleotides having a length of less than 1000 bases are direct copies of the at least one target nucleic acid molecule. In some instances, at least 5%, 10%, 15%, 20%, 25%, 30% or more than 30% of amplicon polynucleotides having a length of no more than 2000 bases are direct copies of the at least one target nucleic acid molecule. In some instances, at least 5%, 10%, 15%, 20%, 25%, 30% or more than 30% of amplicon polynucleotides having a length of 3000-5000 bases are direct copies of the at least one target nucleic acid molecule. In some instances, the ratio of direct copy amplicons to target nucleic acid molecules is at least 10:1, 100:1, 1000:1, 10,000:1, 100,000:1, 1,000,000:1, 10,000,000:1, or more than 10,000,000:1. In some instances, the ratio of direct copy amplicons to target nucleic acid molecules is at least 10:1, 100:1, 1000:1, 10,000:1, 100,000:1, 1,000,000:1, 10,000,000:1, or more than 10,000,000:1, wherein the direct copy amplicons are no more than 700-1200 bases in length. In some instances, the ratio of direct copy amplicons and daughter amplicons to target nucleic acid molecules is at least 10:1, 100:1, 1000:1, 10,000:1, 100,000:1, 1,000,000:1, 10,000,000:1, or more than 10,000,000:1. In some instances, the ratio of direct copy amplicons and daughter amplicons to target nucleic acid molecules is at least 10:1, 100:1, 1000:1, 10,000:1, 100,000:1, 1,000,000:1, 10,000,000:1, or more than 10,000,000:1, wherein the direct copy amplicons are 700-1200 bases in length, and the daughter amplicons are 2500-6000 bases in length. In some instances, the library comprises about 50-10,000, about 50-5,000, about 50-2500, about 50-1000, about 150-2000, about 250-3000, about 50-2000, about 500-2000, or about 500-1500 amplicons which are direct copies of the target nucleic acid molecule. In some instances, the library comprises about 50-10,000, about 50-5,000, about 50-2500, about 50-1000, about 150-2000, about 250-3000, about 50-2000, about 500-2000, or about 500-1500 amplicons which are direct copies of the target nucleic acid molecule or daughter amplicons. The number of direct copies may be controlled in some instances by the number of amplification cycles. In some instances, no more than 30, 25, 20, 15, 13, 11, 10, 9, 8, 7, 6, 5, 4, or 3 cycles are used to generate copies of the target nucleic acid molecule. In some instances, about 30, 25, 20, 15, 13, 11, 10, 9, 8, 7, 6, 5, 4, or about 3 cycles are used to generate copies of the target nucleic acid molecule. In some instances, 3, 4, 5, 6, 7, or 8 cycles are used to generate copies of the target nucleic acid molecule. In some instances, 2-4, 2-5, 2-7, 2-8, 2-10, 2-15, 3-5, 3-10, 3-15, 4-10, 4-15, 5-10 or 5-15 cycles are used to generate copies of the target nucleic acid molecule. Amplicon libraries generated using the methods described herein are in some instances subjected to additional steps, such as adapter ligation and further amplification. In some instances, such additional steps precede a sequencing step. In some instances, the cycles are PCR cycles. In some instances, the cycles represent annealing, extension, and denaturation. In some instances, the cycles represent annealing, extension, and denaturation which occur under isothermal or essentially isothermal conditions.

Methods described herein may additionally comprise one or more enrichment or purification steps. In some instances, one or more polynucleotides (such as cDNA, PTA amplicons, or other polynucleotides) are enriched during a method described herein. In some instances, polynucleotide probes are used to capture one or more polynucleotides. In some instances, probes are configured to capture one or more genomic exons. In some instances, a library of probes comprises at least 1000, 2000, 5000, 10,000, 50,000, 100,000, 200,000, 500,000, or more than 1 million different sequences. In some instances, a library of probes comprises sequences capable of binding to at least 10, 20, 50, 100, 200, 500, 1000, 2000, 5000, 10,000 or more than 10,000 genes. In some instances, probes comprise a moiety for capture by a solid support, such as biotin. In some instances, an enrichment step occurs after a PTA step. In some instances, an enrichment step occurs before a PTA step. In some instances, probes are configured to bind genomic DNA libraries. In some instances, probes are configured to bind cDNA libraries.

Amplicon libraries of polynucleotides generated from the PTA methods and compositions (terminators, polymerases, etc.) described herein in some instances have increased uniformity. Uniformity, in some instances, is described using a Lorenz curve, or other such method. Such increases in some instances lead to lower sequencing reads needed for the desired coverage of a target nucleic acid molecule (e.g., genomic DNA, RNA, or other target nucleic acid molecule). For example, no more than 50% of a cumulative fraction of polynucleotides comprises sequences of at least 80% of a cumulative fraction of sequences of the target nucleic acid molecule. In some instances, no more than 50% of a cumulative fraction of polynucleotides comprises sequences of at least 60% of a cumulative fraction of sequences of the target nucleic acid molecule. In some instances, no more than 50% of a cumulative fraction of polynucleotides comprises sequences of at least 70% of a cumulative fraction of sequences of the target nucleic acid molecule. In some instances, no more than 50% of a cumulative fraction of polynucleotides comprises sequences of at least 90% of a cumulative fraction of sequences of the target nucleic acid molecule. In some instances, uniformity is described using a Gini index (wherein an index of 0 represents perfect equality of the library and an index of 1 represents perfect inequality). In some instances, amplicon libraries described herein have a Gini index of no more than 0.55, 0.50, 0.45, 0.40, or 0.30. In some instances, amplicon libraries described herein have a Gini index of no more than 0.50. In some instances, amplicon libraries described herein have a Gini index of no more than 0.40. Such uniformity metrics in some instances are dependent on the number of reads obtained. For example, no more than 100 million, 200 million, 300 million, 400 million, or no more than 500 million reads are obtained. In some instances, the read length is about 50, 75, 100, 125, 150, 175, 200, 225, or about 250 bases in length. In some instances, uniformity metrics are dependent on the depth of coverage of a target nucleic acid. For example, the average depth of coverage is about 10×, 15×, 20×, 25×, or about 30×. In some instances, the average depth of coverage is 10-30×, 20-50×, 5-40×, 20-60×, 5-20×, or 10-20×. In some instances, amplicon libraries described herein have a Gini index of no more than 0.55, wherein about 300 million reads was obtained. In some instances, amplicon libraries described herein have a Gini index of no more than 0.50, wherein about 300 million reads was obtained. In some instances, amplicon libraries described herein have a Gini index of no more than 0.45, wherein about 300 million reads was obtained. In some instances, amplicon libraries described herein have a Gini index of no more than 0.55, wherein no more than 300 million reads was obtained. In some instances, amplicon libraries described herein have a Gini index of no more than 0.50, wherein no more than 300 million reads was obtained. In some instances, amplicon libraries described herein have a Gini index of no more than 0.45, wherein no more than 300 million reads was obtained. In some instances, amplicon libraries described herein have a Gini index of no more than 0.55, wherein the average depth of sequencing coverage is about 15×. In some instances, amplicon libraries described herein have a Gini index of no more than 0.50, wherein the average depth of sequencing coverage is about 15×. In some instances, amplicon libraries described herein have a Gini index of no more than 0.45, wherein the average depth of sequencing coverage is about 15×. In some instances, amplicon libraries described herein have a Gini index of no more than 0.55, wherein the average depth of sequencing coverage is at least 15×. In some instances, amplicon libraries described herein have a Gini index of no more than 0.50, wherein the average depth of sequencing coverage is at least 15×. In some instances, amplicon libraries described herein have a Gini index of no more than 0.45, wherein the average depth of sequencing coverage is at least 15×. In some instances, amplicon libraries described herein have a Gini index of no more than 0.55, wherein the average depth of sequencing coverage is no more than 15×. In some instances, amplicon libraries described herein have a Gini index of no more than 0.50, wherein the average depth of sequencing coverage is no more than 15×. In some instances, amplicon libraries described herein have a Gini index of no more than 0.45, wherein the average depth of sequencing coverage is no more than 15×. Uniform amplicon libraries generated using the methods described herein are in some instances subjected to additional steps, such as adapter ligation and further PCR amplification. In some instances, such additional steps precede a sequencing step.

Primers comprise nucleic acids used for priming the amplification reactions described herein. Such primers in some instances include, without limitation, random deoxynucleotides of any length with or without modifications to make them exonuclease resistant, random ribonucleotides of any length with or without modifications to make them exonuclease resistant, modified nucleic acids such as locked nucleic acids, DNA or RNA primers that are targeted to a specific genomic region, and reactions that are primed with enzymes such as primase. In the case of whole genome PTA, it is preferred that a set of primers having random or partially random nucleotide sequences be used. In a nucleic acid sample of significant complexity, specific nucleic acid sequences present in the sample need not be known and the primers need not be designed to be complementary to any particular sequence. Rather, the complexity of the nucleic acid sample results in a large number of different hybridization target sequences in the sample, which will be complementary to various primers of random or partially random sequence. The complementary portion of primers for use in PTA are in some instances fully randomized, comprise only a portion that is randomized, or be otherwise selectively randomized. The number of random base positions in the complementary portion of primers in some instances, for example, is from 20% to 100% of the total number of nucleotides in the complementary portion of the primers. In some instances, the number of random base positions in the complementary portion of primers is 10% to 90%, 15-95%, 20%-100%, 30%-100%, 50%-100%, 75-100% or 90-95% of the total number of nucleotides in the complementary portion of the primers. In some instances, the number of random base positions in the complementary portion of primers is at least 10%, 20%, 30%, 40%, 50%, 60%, 70%, 80%, or at least 90% of the total number of nucleotides in the complementary portion of the primers. Sets of primers having random or partially random sequences are in some instances synthesized using standard techniques by allowing the addition of any nucleotide at each position to be randomized. In some instances, sets of primers are composed of primers of similar length and/or hybridization characteristics. In some instances, the term “random primer” refers to a primer which can exhibit four-fold degeneracy at each position. In some instances, the term “random primer” refers to a primer which can exhibit three-fold degeneracy at each position. Random primers used in the methods described herein in some instances comprise a random sequence that is 3, 4, 5, 6, 7, 8, 10, 11, 12, 13, 14, 15, 16, 17, 18, 19, 20, or more bases in length. In some instances, primers comprise random sequences that are 3-20, 5-15, 5-20, 6-12, or 4-10 bases in length. Primers may also comprise non-extendable elements that limit subsequent amplification of amplicons generated thereof. For example, primers with non-extendable elements in some instances comprise terminators. In some instances, primers comprise terminator nucleotides, such as 1, 2, 3, 4, 5, 10, or more than 10 terminator nucleotides. Primers need not be limited to components which are added externally to an amplification reaction. In some instances, primers are generated in-situ through the addition of nucleotides and proteins which promote priming. For example, primase-like enzymes in combination with nucleotides is in some instances used to generate random primers for the methods described herein. Primase-like enzymes in some instances are members of the DnaG or AEP enzyme superfamily. In some instances, a primase-like enzyme is TthPrimPol. In some instances, a primase-like enzyme is T7 gp4 helicase-primase. Such primases are in some instances used with the polymerases or strand displacement factors described herein. In some instances, primases initiate priming with deoxyribonucleotides. In some instances, primases initiate priming with ribonucleotides. In some instances, primers are irreversible primers. In some instances, irreversible primers comprise phosphonothioate linkages.

The PTA amplification can be followed by selection for a specific subset of amplicons. Such selections are in some instances dependent on size, affinity, activity, hybridization to probes, or other known selection factor in the art. In some instances, selections precede or follow additional steps described herein, such as adapter ligation and/or library amplification. In some instances, selections are based on size (length) of the amplicons. In some instances, smaller amplicons are selected that are less likely to have undergone exponential amplification, which enriches for products that were derived from the primary template while further converting the amplification from an exponential into a quasi-linear amplification process. In some instances, amplicons comprising 50-2000, 25-5000, 40-3000, 50-1000, 200-1000, 300-1000, 400-1000, 400-600, 600-2000, or 800-1000 bases in length are selected. Size selection in some instances occurs with the use of protocols, e.g., utilizing solid-phase reversible immobilization (SPRI) on carboxylated paramagnetic beads to enrich for nucleic acid fragments of specific sizes, or other protocol known by those skilled in the art. Optionally or in combination, selection occurs through preferential ligation and amplification of smaller fragments during PCR while preparing sequencing libraries, as well as a result of the preferential formation of clusters from smaller sequencing library fragments during sequencing (e.g., sequencing by synthesis, nanopore sequencing, or other sequencing method) . . . . Other strategies to select for smaller fragments are also consistent with the methods described herein and include, without limitation, isolating nucleic acid fragments of specific sizes after gel electrophoresis, the use of silica columns that bind nucleic acid fragments of specific sizes, and the use of other PCR strategies that more strongly enrich for smaller fragments. Any number of library preparation protocols may be used with the PTA methods described herein. Amplicons generated by PTA are in some instances ligated to adapters (optionally with removal of terminator nucleotides). In some instances, amplicons generated by PTA comprise regions of homology generated from transposase-based fragmentation which are used as priming sites. In some instances, libraries are prepared by fragmenting nucleic acids mechanically or enzymatically. In some instances, libraries are prepared using tagmentation via transposomes. In some instances, libraries are prepared via ligation of adapters, such as Y-adapters, universal adapters, or circular adapters.

The non-complementary portion of a primer used in PTA can include sequences which can be used to further manipulate and/or analyze amplified sequences. An example of such a sequence is a “detection tag”. Detection tags have sequences complementary to detection probes and are detected using their cognate detection probes. There may be one, two, three, four, or more than four detection tags on a primer. There is no fundamental limit to the number of detection tags that can be present on a primer except the size of the primer. In some instances, there is a single detection tag on a primer. In some instances, there are two detection tags on a primer. When there are multiple detection tags, they may have the same sequence or they may have different sequences, with each different sequence complementary to a different detection probe. In some instances, multiple detection tags have the same sequence. In some instances, multiple detection tags have a different sequence.

Another example of a sequence that can be included in the non-complementary portion of a primer is an “address tag” that can encode other details of the amplicons, such as the location in a tissue section. In some instances, a cell barcode comprises an address tag. An address tag has a sequence complementary to an address probe. Address tags become incorporated at the ends of amplified strands. If present, there may be one, or more than one, address tag on a primer. There is no fundamental limit to the number of address tags that can be present on a primer except the size of the primer. When there are multiple address tags, they may have the same sequence or they may have different sequences, with each different sequence complementary to a different address probe. The address tag portion can be any length that supports specific and stable hybridization between the address tag and the address probe. In some instances, nucleic acids from more than one source can incorporate a variable tag sequence. This tag sequence can be up to 100 nucleotides in length, preferably 1 to 10 nucleotides in length, most preferably 4, 5 or 6 nucleotides in length and comprises combinations of nucleotides. In some instances, a tag sequence is 1-20, 2-15, 3-13, 4-12, 5-12, or 1-10 nucleotides in length. For example, if six base-pairs are chosen to form the tag and a permutation of four different nucleotides is used, then a total of 4096 nucleic acid anchors (e.g. hairpins), each with a unique 6 base tag can be made. In some instances, tags identify the source of a sample or analyte. In some instances, tags uniquely identify every molecule in a population.

Primers described herein may be present in solution or immobilized on a solid support. In some instances, primers bearing sample barcodes and/or UMI sequences can be immobilized on a solid support. The solid support can be, for example, one or more beads. In some instances, individual cells are contacted with one or more beads having a unique set of sample barcodes and/or UMI sequences in order to identify the individual cell. In some instances, lysates from individual cells are contacted with one or more beads having a unique set of sample barcodes and/or UMI sequences in order to identify the individual cell lysates. In some instances, extracted nucleic acid from individual cells are contacted with one or more beads having a unique set of sample barcodes and/or UMI sequences in order to identify the extracted nucleic acid from the individual cell. The beads can be manipulated in any suitable manner as is known in the art, for example, using droplet actuators as described herein. The beads may be any suitable size, including for example, microbeads, microparticles, nanobeads and nanoparticles. In some embodiments, beads are magnetically responsive; in other embodiments beads are not significantly magnetically responsive. Non-limiting examples of suitable beads include flow cytometry microbeads, polystyrene microparticles and nanoparticles, functionalized polystyrene microparticles and nanoparticles, coated polystyrene microparticles and nanoparticles, silica microbeads, fluorescent microspheres and nanospheres, functionalized fluorescent microspheres and nanospheres, coated fluorescent microspheres and nanospheres, color dyed microparticles and nanoparticles, magnetic microparticles and nanoparticles, superparamagnetic microparticles and nanoparticles (e.g., DYNABEADS® available from Invitrogen Group, Carlsbad, CA), fluorescent microparticles and nanoparticles, coated magnetic microparticles and nanoparticles, ferromagnetic microparticles and nanoparticles, coated ferromagnetic microparticles and nanoparticles, and those described in U.S. Pat. Appl. Pub. No. US20050260686, US20030132538, US20050118574, 20050277197, 20060159962. Beads may be pre-coupled with an antibody, protein or antigen, DNA/RNA probe or any other molecule with an affinity for a desired target. In some embodiments, primers bearing sample barcodes and/or UMI sequences can be in solution. In certain embodiments, a plurality of droplets can be presented, wherein each droplet in the plurality bears a sample barcode which is unique to a droplet and the UMI which is unique to a molecule such that the UMI are repeated many times within a collection of droplets. In some embodiments, individual cells are contacted with a droplet having a unique set of sample barcodes and/or UMI sequences in order to identify the individual cell. In some embodiments, lysates from individual cells are contacted with a droplet having a unique set of sample barcodes and/or UMI sequences in order to identify the individual cell lysates. In some embodiments, extracted nucleic acid from individual cells are contacted with a droplet having a unique set of sample barcodes and/or UMI sequences in order to identify the extracted nucleic acid from the individual cell.

PTA primers may comprise a sequence-specific or random primer, a cell barcode and/or a unique molecular identifier (UMI) (e.g., linear primer and or hairpin primer). In some instances, a primer comprises a sequence-specific primer. In some instances, a primer comprises a random primer. In some instances, a primer comprises a cell barcode. In some instances, a primer comprises a sample barcode. In some instances, a primer comprises a unique molecular identifier. In some instances, primers comprise two or more cell barcodes. Such barcodes in some instances identify a unique sample source, or unique workflow. Such barcodes or UMIs are in some instances 5, 6, 7, 8, 9, 10, 11, 12, 15, 20, 25, 30, or more than 30 bases in length. Primers in some instances comprise at least 1000, 10,000, 50,000, 100,000, 250,000, 500,000, 10⁶, 10⁷, 10⁸, 10⁹, or at least 10¹⁰ unique barcodes or UMIs. In some instances primers comprise at least 8, 16, 96, or 384 unique barcodes or UMIs. In some instances a standard adapter is then ligated onto the amplification products prior to sequencing; after sequencing, reads are first assigned to a specific cell based on the cell barcode. Suitable adapters that may be utilized with the PTA method include, e.g., xGen® Dual Index UMI adapters available from Integrated DNA Technologies (IDT). Reads from each cell is then grouped using the UMI, and reads with the same UMI may be collapsed into a consensus read. The use of a cell barcode allows all cells to be pooled prior to library preparation, as they can later be identified by the cell barcode. The use of the UMI to form a consensus read in some instances corrects for PCR bias, improving the copy number variation (CNV) detection. In addition, sequencing errors may be corrected by requiring that a fixed percentage of reads from the same molecule have the same base change detected at each position. This approach has been utilized to improve CNV detection and correct sequencing errors in bulk samples. In some instances, UMIs are used with the methods described herein, for example, U.S. Pat. No. 8,835,358 discloses the principle of digital counting after attaching a random amplifiable barcode. Schmitt. et al and Fan et al. disclose similar methods of correcting sequencing errors. In some instances, a library is generated for sequencing using primers. In some instances, the library comprises fragments of 200-700 bases, 100-1000, 300-800, 300-550, 300-700, or 200-800 bases in length. In some instances, the library comprises fragments of at least 50,100,150, 200, 300, 500, 600, 700, 800, or at least 1000 bases in length. In some instances, the library comprises fragments of about 50,100,150, 200, 300, 500, 600, 700, 800, or about 1000 bases in length.

The methods described herein may further comprise additional steps, including steps performed on the sample or template. Such samples or templates in some instance are subjected to one or more steps prior to PTA. In some instances, samples comprising cells are subjected to a pre-treatment step. For example, cells undergo lysis and proteolysis to increase chromatin accessibility using a combination of freeze-thawing, Triton X-100, Tween 20, and Proteinase K. Other lysis strategies are also suitable for practicing the methods described herein. Such strategies include, without limitation, lysis using other combinations of detergent and/or lysozyme and/or protease treatment and/or physical disruption of cells such as sonication and/or alkaline lysis and/or hypotonic lysis. In some instances, the primary template or target molecule(s) is subjected to a pre-treatment step. In some instances, the primary template (or target) is denatured using sodium hydroxide, followed by neutralization of the solution. Other denaturing strategies may also be suitable for practicing the methods described herein. Such strategies may include, without limitation, combinations of alkaline lysis with other basic solutions, increasing the temperature of the sample and/or altering the salt concentration in the sample, addition of additives such as solvents or oils, other modification, or any combination thereof. In some instances, additional steps include sorting, filtering, or isolating samples, templates, or amplicons by size. In some instances, cells are lysed with mechanical (e.g., high pressure homogenizer, bead milling) or non-mechanical (physical, chemical, or biological). In some instances, physical lysis methods comprise heating, osmotic shock, and/or cavitation. In some instances, chemical lysis comprises alkali and/or detergents. In some instances, biological lysis comprises use of enzymes. Combinations of lysis methods are also compatible with the methods described herein. Non-limited examples of lysis enzymes include recombinant lysozyme, serine proteases, and bacterial lysins. In some instances, lysis with enzymes comprises use of lysozyme, lysostaphin, zymolase, cellulose, protease or glycanase. For example, after amplification with the methods described herein, amplicon libraries are enriched for amplicons having a desired length. In some instances, amplicon libraries are enriched for amplicons having a length of 50-2000, 25-1000, 50-1000, 75-2000, 100-3000, 150-500, 75-250, 170-500, 100-500, or 75-2000 bases. In some instances, amplicon libraries are enriched for amplicons having a length no more than 75,100,150, 200, 500, 750, 1000, 2000, 5000, or no more than 10,000 bases. In some instances, amplicon libraries are enriched for amplicons having a length of at least 25, 50, 75, 100, 150, 200, 500, 750, 1000, or at least 2000 bases.

Methods and compositions described herein may comprise buffers or other formulations. Such buffers are in some instances used for PTA, RT, or other method described herein. Such buffers in some instances comprise surfactants/detergent or denaturing agents (Tween-20, DMSO, DMF, pegylated polymers comprising a hydrophobic group, or other surfactant), salts (potassium or sodium phosphate (monobasic or dibasic), sodium chloride, potassium chloride, TrisHCl, magnesium chloride or sulfate, Ammonium salts such as phosphate, nitrate, or sulfate, EDTA), reducing agents (DTT, THP, DTE, beta-mercaptoethanol, TCEP, or other reducing agent) or other components (glycerol, hydrophilic polymers such as PEG). In some instances, buffers are used in conjunction with components such as polymerases, strand displacement factors, terminators, or other reaction component described herein. In some instances, buffers are used in conjunction with components such as polymerases, strand displacement factors, terminators, or other reaction component described herein. Buffers may comprise one or more crowding agents. In some instances, crowding reagents include polymers. In some instances, crowding reagents comprise polymers such as polyols. In some instances, crowding reagents comprise polyethylene glycol polymers (PEG). In some instances, crowding reagents comprise polysaccharides. Without limitation, examples of crowding reagents include ficoll (e.g., ficoll PM 400, ficoll PM 70, or other molecular weight ficoll), PEG (e.g., PEG1000, PEG 2000, PEG4000, PEG6000, PEG8000, or other molecular weight PEG), dextran (dextran 6, dextran 10, dextran 40, dextran 70, dextran 6000, dextran 138k, or other molecular weight dextran).

The nucleic acid molecules amplified according to the methods described herein may be sequenced and analyzed using methods known to those of skill in the art. In some instances, such nucleic acids are obtained from fetal cells. Non-limiting examples of the sequencing methods which in some instances are used include, e.g., sequencing by hybridization (SBH), sequencing by ligation (SBL) (Shendure et al. (2005) Science 309:1728), quantitative incremental fluorescent nucleotide addition sequencing (QIFNAS), stepwise ligation and cleavage, fluorescence resonance energy transfer (FRET), molecular beacons, TaqMan reporter probe digestion, pyrosequencing, fluorescent in situ sequencing (FISSEQ), FISSEQ beads (U.S. Pat. No. 7,425,431), wobble sequencing (Int. Pat. Appl. Pub. No. WO2006/073504), multiplex sequencing (U.S. Pat. Appl. Pub. No. US2008/0269068; Porreca et al., 2007, Nat. Methods 4:931), polymerized colony (POLONY) sequencing (U.S. Pat. Nos. 6,432,360, 6,485,944 and 6,511,803, and Int. Pat. Appl. Pub. No. WO2005/082098), nanogrid rolling circle sequencing (ROLONY) (U.S. Pat. No. 9,624,538), allele-specific oligo ligation assays (e.g., oligo ligation assay (OLA), single template molecule OLA using a ligated linear probe and a rolling circle amplification (RCA) readout, ligated padlock probes, and/or single template molecule OLA using a ligated circular padlock probe and a rolling circle amplification (RCA) readout), high-throughput sequencing methods such as, e.g., methods using Roche 454, Illumina Solexa, AB-SOLID, Helicos, Polonator platforms and the like, and light-based sequencing technologies (Landegren et al. (1998) Genome Res. 8:769-76; Kwok (2000) Pharmacogenomics 1:95-100; and Shi (2001) Clin. Chem. 47:164-172). In some instances, the amplified nucleic acid molecules are shotgun sequenced. Sequencing of the sequencing library is in some instances performed with any appropriate sequencing technology, including but not limited to single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis (array/colony-based or nanoball based). In some instances, sequencing comprises one or more of Sanger sequencing, next generation sequencing, single-molecule real-time sequencing, Polony sequencing, sequencing by synthesis, sequencing by ligation, reversible terminator sequencing, proton detection sequencing, ion semiconductor sequencing, nanopore sequencing, electronic sequencing, pyrosequencing, Maxam-Gilbert sequencing, chain termination sequencing, or +S sequencing.

Sequencing libraries generated using the methods described herein (e.g., PTA or RNAseq) may be sequenced to obtain a desired number of sequencing reads. In some instances, libraries are generated from a single cell or sample comprising a single cell (alone or part of a multiomics workflow). In some instances, libraries are sequenced to obtain at least 0.1, 0.2, 0.4, 0.5, 0.7, 0.8, 0.9, 1, 1.1, 1.2, 1.5, 2, 5, or at least 10 million reads. In some instances, libraries are sequenced to obtain no more than 0.1, 0.2, 0.4, 0.5, 0.7, 0.8, 0.9, 1, 1.1, 1.2, 1.5, 2, 5, or no more than 10 million reads. In some instances, libraries are sequenced to obtain about 0.1, 0.2, 0.4, 0.5, 0.7, 0.8, 0.9, 1, 1.1, 1.2, 1.5, 2, 5, or about 10 million reads. In some instances, libraries are sequenced to obtain 0.1-10, 0.1-5, 0.1-1, 0.2-1, 0.3-1.5, 0.5-1, 1-5, or 0.5-5 million reads per sample. In some instances, the number of reads is dependent on the size of the genome. In some in instances samples comprising bacterial genomes are sequenced to obtain 0.5-1 million reads. In some instances, libraries are sequenced to obtain at least 2, 4, 10, 20, 50, 100, 200, 300, 500, 700, or at least 900 million reads. In some instances, libraries are sequenced to obtain no more than 2, 4, 10, 20, 50, 100, 200, 300, 500, 700, or no more than 900 million reads. In some instances, libraries are sequenced to obtain about 2, 4, 10, 20, 50, 100, 200, 300, 500, 700, or about 900 million reads. In some in instances samples comprising mammalian genomes are sequenced to obtain 500-600 million reads. In some instances, the type of sequencing library (cDNA libraries or genomic libraries) are identified during sequencing. In some instances, cDNA libraries and genomic libraries are identified during sequencing with unique barcodes.

The term “cycle” when used in reference to a polymerase-mediated amplification reaction is used herein to describe steps of dissociation of at least a portion of a double stranded nucleic acid (e.g., a template from an amplicon, or a double stranded template, denaturation). hybridization of at least a portion of a primer to a template (annealing), and extension of the primer to generate an amplicon. In some instances, the temperature remains constant during a cycle of amplification (e.g., an isothermal reaction). In some instances, the number of cycles is directly correlated with the number of amplicons produced. In some instances, the number of cycles for an isothermal reaction is controlled by the amount of time the reaction is allowed to proceed.

High throughput devices and methods described herein may be used for a number of applications. Described herein are methods of identifying mutations in fetal cells using PTA, such as single cells. Use of the PTA method in some instances results in improvements over known methods, for example, MDA. PTA in some instances has lower false positive and false negative variant calling rates than the MDA method. Genomes, such as NA12878 platinum genomes, are in some instances used to determine if the greater genome coverage and uniformity of PTA would result in lower false negative variant calling rate. Without being bound by theory, it may be determined that the lack of error propagation in PTA decreases the false positive variant call rate. The amplification balance between alleles with the two methods is in some cases estimated by comparing the allele frequencies of the heterozygous mutation calls at known positive loci. In some instances, amplicon libraries generated using PTA are further amplified by PCR. In some instances, PTA is used in a workflow with additional analysis methods, such as RNAseq, methylome analysis or other method described herein.

Described herein are methods of measuring mutagenicity of an environmental factor in fetal cells. For example, cells (such as fetal single cells or a population of fetal cells) are exposed to a potential environmental condition. In some instances, the cells are exposed to a potential environmental condition via the mother. In some instances, an environmental condition comprises heat, light (e.g. ultraviolet), radiation, a chemical substance, or any combination thereof. After an amount of exposure to the environmental condition, in some instances minutes, hours, days, or longer, single cells are isolated and subjected to the PTA method. In some instances, molecular barcodes and unique molecular identifiers are used to tag the sample. The sample is sequenced and then analyzed to identify gene expression alterations and or resulting from mutations resulting from exposure to the environmental condition. In some instances, such mutations are compared with a control environmental condition, such as a known non-mutagenic substance, vehicle/solvent, or lack of an environmental condition. Such analysis in some instances not only provides the total number of mutations caused by the environmental condition, but also the locations and nature of such mutations. Patterns are in some instances identified from the data and may be used for diagnosis of diseases or conditions. In some instances, patterns are used to predict future disease states or conditions. In some instances, the methods described herein measure the mutation burden, locations, and patterns in a cell after exposure to an environmental agent, such as, e.g., a potential mutagen or teratogen. This approach in some instances is used to evaluate the safety of a given agent, including its potential to induce mutations that can contribute to the development of a disease. For example, the method could be used to predict the carcinogenicity or teratogenicity of an agent to specific cell types after exposure to a specific concentration of the specific agent.

Described herein are methods of determining gene expression alteration in combination with the mutations in cells that are used for cellular therapy, such as but not limited to the transplantation of induced pluripotent stem cells, transplantation of hematopoietic or other cells that have not be manipulated, or transplantation of hematopoietic or other cells that have undergone genome edits. The cells can then undergo PTA and sequencing to determine mutation burden and mutation combination in each cell. The per-cell mutation rate and locations of mutations in the cellular therapy product can be used to assess the safety and potential efficacy of the product.

Cells for use with the PTA method may be fetal cells, such as embryonic cells. In some embodiments, PTA is used in conjunction with non-invasive preimplantation genetic testing (NIPGT). In a further embodiment, cells can be isolated from blastomeres that are created by in vitro fertilization. The cells can then undergo PTA and sequencing to determine the burden and combination of potentially disease predisposing genetic variants in each cell. The gene expression alteration in combination with the mutation profile of the cell can then be used to extrapolate the genetic predisposition of the blastomere to specific diseases prior to implantation. In some instances embryos in culture shed nucleic acids that are used to assess the health of the embryo using low pass genome sequencing. In some instances, embryos are frozen-thawed. In some instances, nucleic acids are obtained from blastocyte culture conditioned medium (BCCM), blastocoel fluid (BF), or a combination thereof. In some instances, PTA analysis of fetal cells is used to detect chromosomal abnormalities, such as fetal aneuploidy. In some instances, PTA is used to detect diseases such as Down's or Patau syndromes. In some instances, frozen blastocytes are thawed and cultured for a period of time before obtaining nucleic acids for analysis (e.g., culture media, BF, or a cell biopsy). In some instances, blastocytes are cultured for no more than 4, 6, 8, 12, 16, 24, 36, 48, or no more than 64 hours prior to obtaining nucleic acids for analysis.

### Definitions

Unless defined otherwise, all technical and scientific terms used herein have the same meaning as is commonly understood by one of ordinary skill in the art to which these inventions belong.

Throughout this disclosure, numerical features are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

The terms “subject” or “patient” or “individual”, as used herein, refer to animals, including mammals, such as, e.g., humans, veterinary animals (e.g., cats, dogs, cows, horses, sheep, pigs, etc.) and experimental animal models of diseases (e.g., mice, rats). In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, New York (herein “Sambrook et al., 1989”); DNA Cloning: A practical Approach, Volumes I and II (D. N. Glover ed. 1985); Oligonucleotide Synthesis (M J. Gait ed. 1984); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; B. Perbal, A practical Guide To Molecular Cloning (1984); F. M. Ausubel et al. (eds.), Current Protocols in Molecular Biology, John Wiley & Sons, Inc. (1994); among others.

The term “nucleic acid” encompasses multi-stranded, as well as single-stranded molecules. In double- or triple-stranded nucleic acids, the nucleic acid strands need not be coextensive (i.e., a double-stranded nucleic acid need not be double-stranded along the entire length of both strands). Nucleic acid templates described herein may be any size depending on the sample (from small cell-free DNA fragments to entire genomes), including but not limited to 50-300 bases, 100-2000 bases, 100-750 bases, 170-500 bases, 100-5000 bases, 50-10,000 bases, or 50-2000 bases in length. In some instances, templates are at least 50,100,200, 500, 1000, 2000, 5000, 10,000, 20,000 50,000, 100,000, 200,000, 500,000, 1,000,000 or more than 1,000,000 bases in length. Methods described herein provide for the amplification of nucleic acid acids, such as nucleic acid templates. Methods described herein additionally provide for the generation of isolated and at least partially purified nucleic acids and libraries of nucleic acids. In some instances, methods described herein provide for extracted nucleic acids (e.g., extracted from tissues, cells, or media). Nucleic acids include but are not limited to those comprising DNA, RNA, circular RNA, mtDNA (mitochondrial DNA), cfDNA (cell free DNA), cfRNA (cell free RNA), siRNA (small interfering RNA), cffDNA (cell free fetal DNA), mRNA, tRNA, rRNA, miRNA (microRNA), synthetic polynucleotides, polynucleotide analogues, any other nucleic acid consistent with the specification, or any combinations thereof. The length of polynucleotides, when provided, are described as the number of bases and abbreviated, such as nt (nucleotides), bp (bases), kb (kilobases), or Gb (gigabases).

The term “droplet” as used herein refers to a volume of liquid on a droplet actuator. Droplets in some instances, for example, be aqueous or non-aqueous or may be mixtures or emulsions including aqueous and non-aqueous components. For non-limiting examples of droplet fluids that may be subjected to droplet operations, see, e.g., Int. Pat. Appl. Pub. No. WO2007/120241. Any suitable system for forming and manipulating droplets can be used in the embodiments presented herein. For example, in some instances a droplet actuator is used. For non-limiting examples of droplet actuators which can be used, see, e.g., U.S. Pat. Nos. 6,911,132, 6,977,033, 6,773,566, 6,565,727, 7,163,612, 7,052,244, 7,328,979, 7,547,380, 7,641,779, U.S. Pat. Appl. Pub. Nos. US20060194331, US20030205632, US20060164490, US20070023292, US20060039823, US20080124252, US20090283407, US20090192044, US20050179746, US20090321262, US20100096266, US20110048951, Int. Pat. Appl. Pub. No. WO2007/120241. In some instances, beads are provided in a droplet, in a droplet operations gap, or on a droplet operations surface. In some instances, beads are provided in a reservoir that is external to a droplet operations gap or situated apart from a droplet operations surface, and the reservoir may be associated with a flow path that permits a droplet including the beads to be brought into a droplet operations gap or into contact with a droplet operations surface. Non-limiting examples of droplet actuator techniques for immobilizing magnetically responsive beads and/or non-magnetically responsive beads and/or conducting droplet operations protocols using beads are described in U.S. Pat. Appl. Pub. No. US20080053205, Int. Pat. Appl. Pub. No. WO2008/098236, WO2008/134153, WO2008/116221, WO2007/120241. Bead characteristics may be employed in the multiplexing embodiments of the methods described herein. Examples of beads having characteristics suitable for multiplexing, as well as methods of detecting and analyzing signals emitted from such beads, may be found in U.S. Pat. Appl. Pub. No. US20080305481, US20080151240, US20070207513, US20070064990, US20060159962, US20050277197, US20050118574.

Primers and/or template switching oligonucleotides can also be affixed to solid substrate to facilitate reverse transcription and template switching of the mRNA polynucleotides. In this arrangement a portion of the RT or template switching reaction occurs in the bulk solution of the device, where the second step of the reaction occurs in proximity to the surface. In other arrangements the primer of template switch oligonucleotide is allowed to be released from the solid substrate to allow the entire reaction to occur above the surface in the solution. In a polyomic approach the primers for the multistage reaction in some instances is affixed to the solid substrate or combined with beads to accomplish combinations of multistage primers.

Certain microfluidic devices also support polyomic approaches. Devices fabricated in PDMS, as an example, often have contiguous chambers for each reaction step. Such multichambered devices are often segregated using a microvalve structure which can be controlled though the pressure with air, or a fluid such as water or inert hydrocarbon (i.e. fluorinert). In a multiomic approach each stage of the reaction can be sequestered and allowed to be conducted discretely. At the completion of a particular stage a valve between an adjacent chamber can be released on the substrates for the subsequent reaction can be added in a serial fashion. The result is the ability to emulate an sequential set of reactions, such as a multiomic (Protein/RNA/DNA/epigenomic) set of reactions using an individual cell as a input template material. Various microfluidics platforms may be used for analysis of single cells. Cells in some instances are manipulated through hydrodynamics (droplet microfluidics, inertial microfluidics, vortexing, microvalves, microstructures (e.g., microwells, microtraps)), electrical methods (dielectrophoresis (DEP), electroosmosis), optical methods (optical tweezers, optically induced dielectrophoresis (ODEP), opto-thermocapillary), acoustic methods, or magnetic methods. In some instances, the microfluidics platform comprises microwells. In some instances, the microfluidics platform comprises a PDMS (Polydimethylsiloxane)-based device. Non-limited examples of single cell analysis platforms compatible with the methods described herein are: ddSEQ Single-Cell Isolator, (Bio-Rad, Hercules, CA, USA, and Illumina, San Diego, CA, USA)); Chromium (10× Genomics, Pleasanton, CA, USA)); Rhapsody Single-Cell Analysis System (BD, Franklin Lakes, NJ, USA); Tapestri Platform (MissionBio, San Francisco, CA, USA)), Nadia Innovate (Dolomite Bio, Royston, UK); C1 and Polaris (Fluidigm, South San Francisco, CA, USA); ICELL8 Single-Cell System (Takara); MSND (Wafergen); Puncher platform (Vycap); CellRaft AIR System (CellMicrosystems); DEPArray NxT and DEP Array System (Menarini Silicon Biosystems); AVISO CellCelector (ALS); and InDrop System (1CellBio), and TrapTx (Celldom).

As used herein, the term “unique molecular identifier (UMI)” refers to a unique nucleic acid sequence that is attached to each of a plurality of nucleic acid molecules. When incorporated into a nucleic acid molecule, an UMI in some instances is used to correct for subsequent amplification bias by directly counting UMIs that are sequenced after amplification. The design, incorporation and application of UMIs is described, for example, in Int. Pat. Appl. Pub. No. WO 2012/142213, Islam et al. Nat. Methods (2014) 11:163-166, Kivioja, T. et al. Nat. Methods (2012) 9:72-74, Brenner et al. (2000) PNAS 97(4), 1665, and Hollas and Schuler, (2003) Conference: 3rd International Workshop on Algorithms in Bioinformatics, Volume: 2812.

As used herein, the term “barcode” refers to a nucleic acid tag that can be used to identify a sample or source of the nucleic acid material. Thus, where nucleic acid samples are derived from multiple sources, the nucleic acids in each nucleic acid sample are in some instances tagged with different nucleic acid tags such that the source of the sample can be identified. Barcodes, also commonly referred to indexes, tags, and the like, are well known to those of skill in the art. Any suitable barcode or set of barcodes can be used. See, e.g., non-limiting examples provided in U.S. Pat. No. 8,053,192 and Int. Pat. Appl. Pub. No. WO2005/068656. Barcoding of single cells can be performed as described, for example, in U.S. Pat. Appl. Pub. No. 2013/0274117.

The terms “solid surface,” “solid support” and other grammatical equivalents herein refer to any material that is appropriate for or can be modified to be appropriate for the attachment of the primers, barcodes and sequences described herein. Exemplary substrates include, but are not limited to, glass and modified or functionalized glass, plastics (including acrylics, polystyrene and copolymers of styrene and other materials, polypropylene, polyethylene, polybutylene, polyurethanes, Teflon™, etc.), polysaccharides, nylon, nitrocellulose, ceramics, resins, silica, silica-based materials (e.g., silicon or modified silicon), carbon, metals, inorganic glasses, plastics, optical fiber bundles, and a variety of other polymers. In some embodiments, the solid support comprises a patterned surface suitable for immobilization of primers, barcodes and sequences in an ordered pattern.

As used herein, the term “biological sample” includes, but is not limited to, tissues, cells, biological fluids and isolates thereof. Cells or other samples used in the methods described herein are in some instances isolated from human patients, animals, plants, soil or other samples comprising microbes such as bacteria, fungi, protozoa, etc. In some instances, the biological sample is of human origin. In some instances, the biological is of non-human origin. The cells in some instances undergo PTA methods described herein and sequencing. Variants detected throughout the genome or at specific locations can be compared with all other cells isolated from that subject to trace the history of a cell lineage for research or diagnostic purposes. In some instances, variants are confirmed through additional methods of analysis such as direct PCR sequencing.

### Embodiments

Also described herein are the following embodiments:
1. A method of embryonic nucleic acid sample preparation useful for determining the presence or absence of fetal genetic abnormalities comprising:
   a. isolating nucleic acids from at least one embryonic cell;
   b. subjecting the nucleic acids to a sample workflow; and
   c. determining if the embryonic cell comprises at least two fetal genetic abnormalities by analyzing the nucleic acids from the sample workflow, wherein the fetal genetic abnormalities comprise:
      i. at least one copy number variation; and
      ii. at least one single nucleotide variant.
2. The method of embodiment 1, wherein the embryonic cell comprises a preimplantation embryonic cell, a blastocyte cell, blastomere cell, a cell obtained from the trophectoderm, a placental cell, or a cell derived from extra-embryonic membranes.
3. The method of embodiment 1 or 2, wherein the embryonic cell comprises a preimplantation embryonic cell.
4. The method of any one of embodiments 1-3, wherein the fetal genetic abnormality comprises two or more of aneuploidy, monogenic disorders, and structural rearrangements.
5. The method of any one of embodiments 1-4, wherein the fetal genetic abnormality comprises aneuploidy, monogenic disorders, and structural rearrangements.
6. The method of any one of embodiments 1-5, wherein determining comprises obtaining information on fetal genetic abnormalities identifiable by PGT-A, PGT-M, or PGT-SR testing.
7. The method of any one of embodiments 1-6, wherein the genetic abnormality comprises phenylketonuria (PKU), sickle-cell anemia, Beta Thalassemia, Tay-Sachs disease, Sandhoff disease, or cystic fibrosis (CF).
8. The method of any one of embodiments 1-6 wherein the genetic abnormality comprises achondroplasia, congenital adrenal hyperplasia, Cystic fibrosis, Down syndrome, fragile XD syndrome, Hemophilia A, Huntington's disease, Muscular dystrophy, Polycystic kidney disease, Sickle cell disease, Tay-Sachs disease, trisomy 21, trisomy 18, trisomy 13, Turner syndrome, spina bifida, anencephaly, or Thalassemia.
9. The method of any one of embodiments 4, wherein the aneuploidy comprises monosomy, trisomy, triploidy, deletions, duplications, or uniparental disomy.
10. The method of embodiment 9, wherein the uniparental disomy occurs at least in four chromosomes.
11. The method of embodiment 9, wherein the uniparental disomy occurs at chromosomes 6, 7, 11, 14, or 15.
12. The method of any one of embodiments 1-11, wherein the fetal genetic abnormality comprises an insertion, deletion or duplication.
13. The method of embodiment 12, wherein the insertion, deletion or duplication is at least 5% of the total chromosome length.
14. The method of embodiment 12 or 13, wherein the insertion, deletion or duplication is less than 15% of the total chromosome length.
15. The method of any one of embodiments 1-14, wherein the method further comprises obtaining the cell from at least a 5 day old blastocyte.
16. The method of any one of embodiments 1-15, comprising at least 4 embryonic cells.
17. The method of any one of embodiments 1-16, wherein a fetal genetic abnormality is detected in no more than 30% of the embryonic cells.
18. The method of any one of embodiments 1-17, wherein a fetal genetic abnormality is detected in 30%-100% of the embryonic cells.
19. The method of any one of embodiments 1-18, wherein the method further comprises obtaining the embryonic cell from a location proximal to an external os of a uterine cervix or anywhere within the vaginal canal of a subject.
20. The method of any one of embodiments 1-19, wherein the method further comprises obtaining the embryonic cell from a Pap smear.
21. The method of any one of embodiments 1-20, wherein the embryonic cell is human.
22. The method of any one of embodiments 1-21, comprising 6-200 embryonic cells.
23. The method of embodiment 22, further comprising measuring a level of mosaicism for the embryonic cells.
24. The method of any one of embodiments 1-23, further comprising establishing the presence or absence of sex chromosomes in the embryonic cell.
25. The method of any one of embodiments 1-24, wherein the embryonic cell is a preimplantation embryonic cell from an embryo, and the method further comprises implanting the embryo in a female.
26. The method of any one of embodiments 1-25, wherein the fetal genetic abnormalities are determined without a blood or saliva test.
27. The method of any one of embodiments 1-26, wherein the fetal genetic abnormalities are determined without amniocentesis, chorionic villus sampling, or Percutaneous umbilical blood sampling.
28. The method of any one of embodiments 1-27, wherein determining comprises sequencing the nucleic acids.
29. The method of embodiment 28, wherein sequencing comprises Sanger sequencing, next generation sequencing, single-molecule real-time sequencing, Polony sequencing, sequencing by synthesis, sequencing by ligation, reversible terminator sequencing, proton detection sequencing, ion semiconductor sequencing, nanopore sequencing, electronic sequencing, pyrosequencing, Maxam-Gilbert sequencing, chain termination sequencing, or +S sequencing.
30. The method of embodiments 28 or 29, wherein the method further comprises exome capture prior to sequencing.
31. The method of any one of embodiments 1-30, wherein the sample workflow comprises:
   contacting the nucleic acids with at least one amplification primer, at least one nucleic acid polymerase, and a mixture of nucleotides, wherein the mixture of nucleotides comprises at least one terminator nucleotide which terminates nucleic acid replication by the polymerase, and
   amplifying at least some of the nucleic acids to generate a plurality of terminated amplification products, wherein the replication proceeds by strand displacement replication.
32. A method of embryonic nucleic acid sample preparation useful for determining the presence or absence of fetal genetic abnormalities comprising:
   a. isolating at least one embryonic cell from a plurality of cells, wherein the plurality of cells comprises fetal and maternal cells;
   b. isolating nucleic acids from the at least one embryonic cell;
   c. contacting the nucleic acids with at least one amplification primer, at least one nucleic acid polymerase, and a mixture of nucleotides, wherein the mixture of nucleotides comprises at least one terminator nucleotide which terminates nucleic acid replication by the polymerase;
   d. amplifying at least some of the nucleic acids to generate a plurality of terminated amplification products, wherein the replication proceeds by strand displacement replication; and
   e. determining if the embryonic cell comprises one or more genetic abnormalities by analyzing the terminated amplification products.
33. The method of embodiment 32, wherein the embryonic cell is isolated from the plurality of cells on a surface.
34. The method of embodiment 32 or 33, wherein the fetal cells are obtained from one or more of the trophectoderm, placenta, or extra-embryonic membranes.
35. The method of any one of embodiments 32-34, wherein the embryonic cell is isolated by an automated robotic device.
36. The method of embodiment 35, wherein the robotic device comprises a capillary fitting.
37. The method of embodiment 32, wherein the embryonic cell is uniquely identified from other non-embryonic cells.
38. The method of embodiment 32, wherein the embryonic cell is uniquely identified from other non-embryonic cells by labeling.
39. The method of embodiment 38, wherein labeling comprises contacting the plurality of cells with an antibody.
40. The method of embodiment 39, wherein the antibody is configured to bind selectively to non-fetal cells.
41. The method of embodiment 39, wherein the antibody is configured to bind selectively to fetal cells.
42. The method of embodiment 39, wherein the antibody is configured to bind to HLA-G or βhCG.
43. The method of any one of embodiments 32-42, wherein isolating comprises FACS sorting.
44. The method of embodiment 39, wherein the antibody comprises a magnetic nanoparticle.
45. The method of any one of embodiments 32-45, wherein the embryonic cell is removed as early as 5 weeks after pregnancy.
46. The method of any one of embodiments 32-45, wherein the embryonic cell is removed as early as 8 weeks after pregnancy.
47. The method of any one of embodiments 32-47, wherein determining comprises in-silico removal of maternal nucleic acid sequences.
48. The method of any one of embodiments 32-48, wherein the terminator is an irreversible terminator.
49. The method of any one of embodiments 32-48, wherein the terminator nucleotide is selected from the group consisting of nucleotides with modification to the alpha group, C3 spacer nucleotides, locked nucleic acids (LNA), inverted nucleic acids, 2′ fluoro nucleotides, 3′ phosphorylated nucleotides, 2′-O-Methyl modified nucleotides, and trans nucleic acids.
50. The method of embodiment 50, wherein the nucleotides with modification to the alpha group are alpha-thio dideoxynucleotides.
51. The method of any one of embodiments 32-51, wherein the terminator nucleotide comprises modifications of the r group of the 3′ carbon of the deoxyribose.
52. The method of any one of embodiments 32-52, wherein the terminator nucleotide is selected from the group consisting of dideoxynucleotides, inverted dideoxynucleotides, 3′ biotinylated nucleotides, 3′ amino nucleotides, 3′-phosphorylated nucleotides, 3′-O-methyl nucleotides, 3′ carbon spacer nucleotides including 3′ C3 spacer nucleotides, 3′ C18 nucleotides, 3′ Hexanediol spacer nucleotides, acyclonucleotides, and combinations thereof.
53. The method of any one of embodiments 32-53, wherein the plurality of terminated amplification products comprise an average of 1000-2000 bases in length.
54. The method of any one of embodiments 32-54, wherein at least some of the amplification products comprise a cell barcode or a sample barcode.
55. The method of any one of embodiments 32-55, wherein amplification occurs for at least five cycles.

### EXAMPLES

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### Example 1: Primary Template-Directed Amplification

Single Cell Capture by FACS Sorting. A low bind 96-well PCR plate was placed on a PCR cooler. 3 μL of Cell Buffer was added to all the wells where cells will be sorted. Single cells were deposited into individual wells of the 96-well plate using fluorescent activated cell sorting (FACS). After single cell sorting, the plate was sealed. The plate was mixed for 10 seconds at 1400 RPM on a PCR Plate Thermal Mixer at room temperature, spun briefly, and placed on ice. Alternatively, plates containing sorted cells were stored on dry ice or at −80° C. with a seal until ready.

Single Cell Whole Genome Amplification with PTA. Reactions were assembled in a DNA-free pre-PCR hood. All reagents were thawed on ice until ready to use. Before use, each reagent was vortexed for 10 sec and spun briefly. Reagents were dispensed to the wall of the tube without touching cell suspension. 96-well PCR plate containing cells were placed on the PCR cooler. If cells were stored at −80° C., cells were thawed on ice for 5 minutes, spun for 10 seconds, then the plate was placed on the PCR cooler (or ice). 1× Reagent Mix was prepared by diluting 12× mix, mixing on the vortexer, and briefly spinning the tube. MS Mix was prepared by combining 1× reagent mix and lysis buffer, mixing on the vortexer, and briefly spinning the tube. 3 μL of MS Mix was added to each well of the plate, and the plate was sealed with the sealing film. After spinning for 10 sec, mixing at room temperature for 1 min at 1400 rpm (plate mixer), and spinning for 10 sec, the plate was placed back on PCR cooler (or ice) for 10 minutes. 3 μL of neutralization buffer was then added, and the plate was sealed with the plate film. After spinning for 10 sec, mixing at room temperature for 1 min at 1400 rpm (plate mixer), spinning for 10 sec, the plate was placed back on the PCR cooler. 3 μL of buffer was added, and the plate was sealed with the plate film. Next, the plate was spun for 10 sec, mixed at room temperature for 1 min at 1400 rpm (plate mixer), and spun for 10 sec followed by incubating at room temperature for 10 min. During the incubation step, the Reaction Mix was prepared by combining the components in the order (nucleotide/terminator reagents, 5.0 μL; 1× reagent mix, 1.0 μL; Phi20 polymerase, 0.8 μL; singe-stranded binding protein reagent, 1.2 μL), followed by mixing gently and thoroughly by pipetting up and down 10 times, then spun briefly. When the incubation was completed, the plate was placed on the PCR cooler (or ice). 8 μL of Reaction Mix was added to each sample while the plate was still on the PCR cooler (or ice), and mixed at room temperature for 1 min at 1000 rpm in plate mixer, then spun briefly. The plate was placed on a thermal cycler (lid set to 70° C.) with the following program: 30° C. for 10 hrs, 65° C. for 3 min, 4° C. hold.

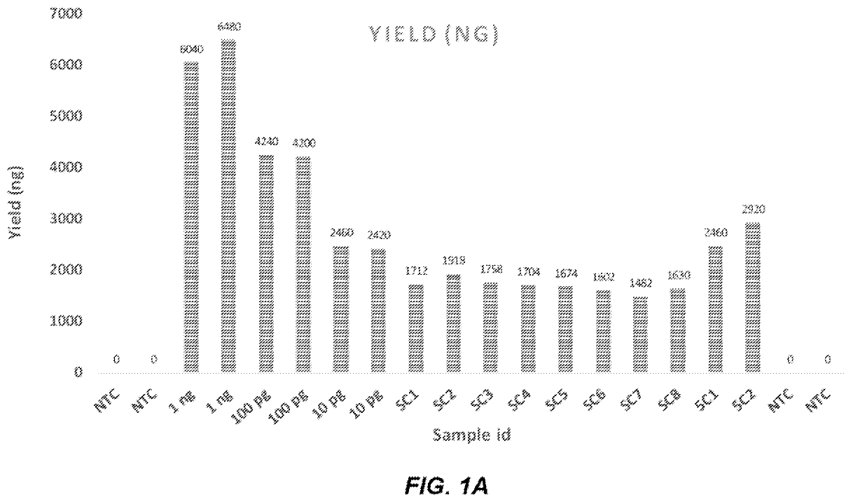

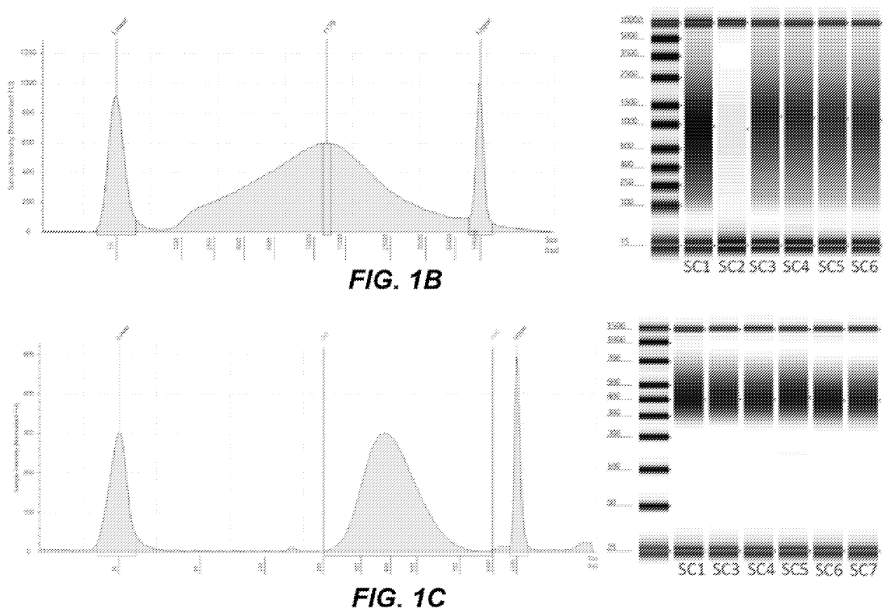

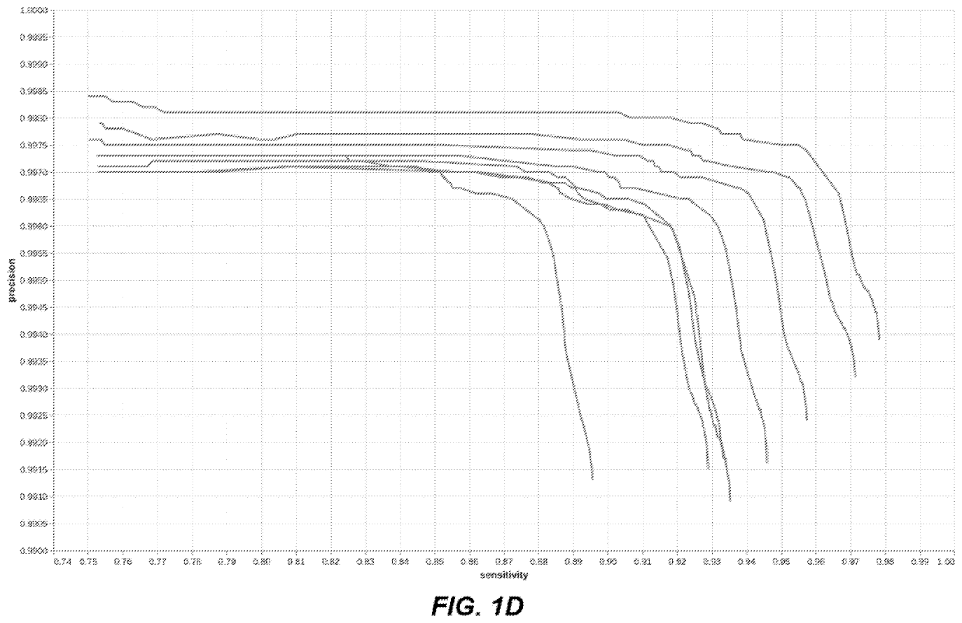

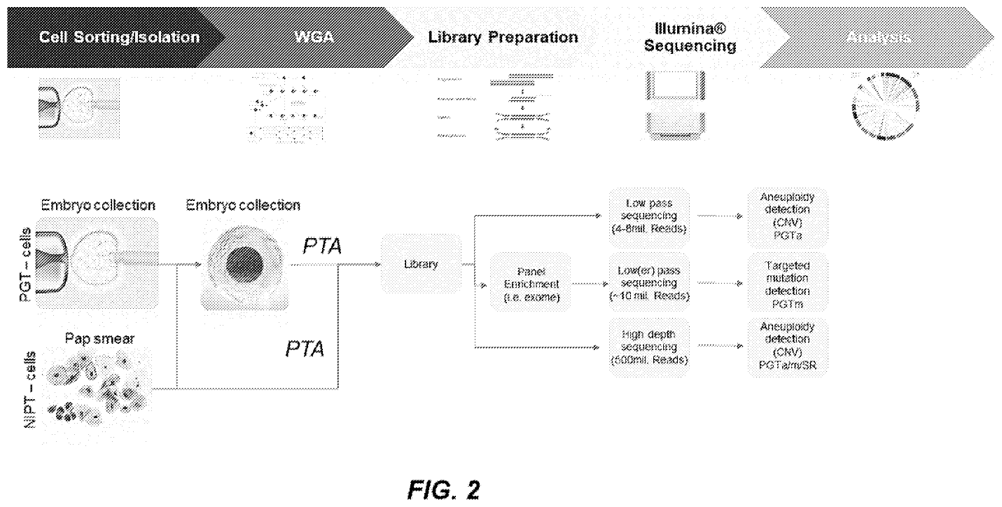

Amplified DNA Cleanup. Capture beads were allowed to equilibrate to room temperature for 30 min. Beads are mixed thoroughly, and then 40 μL of beads were added to each reaction well (vortex and spin). Beads were aspirated prior to each dispensing step, incubated at room temperature for 10 minutes, and the sample plate briefly centrifuged. The plate was placed on a magnet for 3 minutes or until the supernatant cleared. While on the magnet, the supernatant was removed and discarded, being careful not to disturb the beads containing DNA. While on the magnet, 200 μL of freshly prepared 80% ethanol was added to the beads and incubated for 30 seconds at room temperature. While still on the magnet, the first ethanol wash was removed and discarded, taking care not to disturb the beads. Another 200 μL of freshly prepared 80% ethanol was added to the beads, and then incubated for 30 seconds at room temperature. The second ethanol wash was then removed and discarded, taking care not to disturb the beads. Any remaining ethanol from the wells was discarded. The beads were then incubated at room temperature for 5 minutes to air-dry beads, then the plate was removed from the magnet. Beads were then re-suspended in 40 μL of elution buffer, incubated for 2 minutes at room temperature, and placed on the magnet for 3 minutes, or until the supernatant clears. 38 μL of the eluted DNA was transferred to a new plate, for DNA quantification. DNA was then ready to use in downstream applications such as PCR or Real Time PCR. Results are shown in FIG. 1A.

DNA Quantification. Quantitate DNA using the High Sensitivity dsDNA Assay kit (Qubit) as per manufacturer. Size fragment analysis was completed to ensure proper amplification product size. Fragment size distribution was determined by running 1 μL of PTA product on an E-Gel EX, or 1 μL of 2 ng/μL in a High Sensitivity Bioanalyzer DNA Chip. Results are shown in FIG. 1B.

End Repair and A-tailing. 500 ng of amplified DNA was added to a PCR tube. DNA volume was adjusted to 35 μL with RT-PCR grade water. The End-Repair A-Tail Reaction was assembled on a PCR cooler (or ice) as follows: Amplified DNA (500 ng total DNA/Rxn, 35 μL), RT-PCR grade water (10 μL), fragmentation buffer (5 μL), ER/AT buffer (7 μL), ER/AT enzyme (3 μL) to a total volume of 60 μL, which was mixed thoroughly and spun briefly. The mixture was then incubated at 65° C. on a thermal cycler with the lid at 105° C. for 30 minutes.

Adapter Ligation. Multi-Use Library Adapters stock plate was diluted to 1× by adding 54 μL of 10 mM Tris-HCl, 0.1 mM EDTA, pH 8.0 to each well. In the same plate/tube(s) in which end-repair and A-tailing was performed, each Adapter Ligation Reaction was assembled as follows: ER/AT DNA (60 μL), 1× Multi-Use Library Adapters (5 μL), RT-PCR grade water (5 μL), ligation buffer (30 μL), and DNA ligase (10 μL) to a total volume of 110 μL. After thorough mixing and brief spin, the mixture was incubated at 20° C. on thermal cycler for 15 minutes (heated lid not required).

Post Ligation Cleanup. Beads were allowed to equilibrate to room temperature for 30 minutes then mixed thoroughly and immediately before pipetting. In the same plate/tube(s), a 0.8×SPRI cleanup was assembled as follows: adapter-ligated DNA (110 μL), and beads (88 μL) to a final volume of 198 μL. The mixture was mixed thoroughly and incubated for 10 min at room temperature, and the plate/tube(s) are placed on the magnet for 2 minutes, or until the supernatant clears. While on the magnet, the supernatant was removed and discarded being careful not to disturb any beads, followed by washing with 200 μL of freshly prepared 80% ethanol to the beads and incubating for 30 seconds at room temperature. While still on the magnet, the first ethanol wash was removed and discarded, taking care not to disturb the beads. Another 200 μL of freshly prepared 80% ethanol was added to the beads, and then incubated for 30 seconds at room temperature. The second ethanol wash was then removed and discarded, taking care not to disturb the beads. Any remaining ethanol from the wells was discarded. The beads were then incubated at room temperature for 5 minutes to air-dry beads, then the plate was removed from the magnet. Beads were then re-suspended in 20 μL of elution buffer, incubated for 2 minutes at room temperature, and placed on the magnet for 3 minutes, or until the supernatant clears.

Library Amplification. In the same plate/tube(s) containing the DNA-Bead slurry, each library amplification reaction was assembled as follows: adapter ligated library (20 μL), 10×KAPA library amplification primer mix (5 μL), and 2×KAPA HiFi Hotstart ready mix (25 μL) to a total volume of 50 μL. After mixing thoroughly and spinning briefly, amplification was conducted using the cycling protocol: Initial Denaturation 98° C. @ 45 sec (1 cycle), Denaturation 98° C. @ 15 sec; Annealing 60° C. 30 sec; and Extension 72° C. 30 sec (10 cycles), Final Extension 72° C. @ 1 min for 1 cycle, and HOLD 4° C. indefinitely. The heated lid was set to 105° C. The plate/tube(s) were stored at 4° C. for up to 72 hours, or directly used for Post-Amplification Cleanup.

Post Amplification Clean up. Beads were allowed to equilibrate to room temperature for 30 minutes. Beads were mixed thoroughly and immediately before pipetting, and in the same plate/tube(s), a 0.55×SPRI cleanup was assembled as follows: amplified library (50.0 μL) and beads (27.5 μL) to a total volume of 77.5 μL, followed by thorough mixing and incubation for 10 min at room temperature. Plate/tube(s) were placed on the magnet for 3 minutes, or until the supernatant clears. While on the magnet, the supernatant was transferred to a new plate/tube(s) being careful not to transfer any beads.

In a plate/tube(s), a 0.25×SPRI cleanup was assembled as follows: 0.55× Cleanup Supernatant (77.5 μL), and beads (12.5 μL) to a total volume of 90.0 μL. After thorough mixing, the mixture was spun down and incubated for 10 min at room temperature. Plate/tube(s) were placed on the magnet for 3 minutes or until the supernatant clears. While on the magnet, the supernatant was removed and discarded being careful not to disturb any beads, followed by washing with 200 μL of freshly prepared 80% ethanol to the beads and incubating for 30 seconds at room temperature. While still on the magnet, the first ethanol wash was removed and discarded, taking care not to disturb the beads. Another 200 μL of freshly prepared 80% ethanol was added to the beads, and then incubated for 30 seconds at room temperature. The second ethanol wash was then removed and discarded, taking care not to disturb the beads. Any remaining ethanol from the wells was discarded. The beads were then incubated at room temperature for 5 minutes to air-dry beads, then the plate was removed from the magnet. Beads were then re-suspended in 42 μL of elution buffer, incubated for 2 minutes at room temperature, and placed on the magnet for 3 minutes, or until the supernatant clears. 40 μL of the eluted DNA was transferred to a new plate, for DNA quantification.

Library Quantification. The amplified library was quantitated using a Qubit dsDNA kit as per manufacturer. Fragment size distribution was determined by running 1 μL of library on an E-Gel EX, or 1 μL of 2 ng/μL in a Bioanalyzer DNA Chip. Results are shown in FIG. 1C.

Using this workflow, sensitivity vs. precision of SNV calling in GM12878 single cells was measured (FIG. 1D).

### Example 2: Analysis of Embryonic Cells with PTA

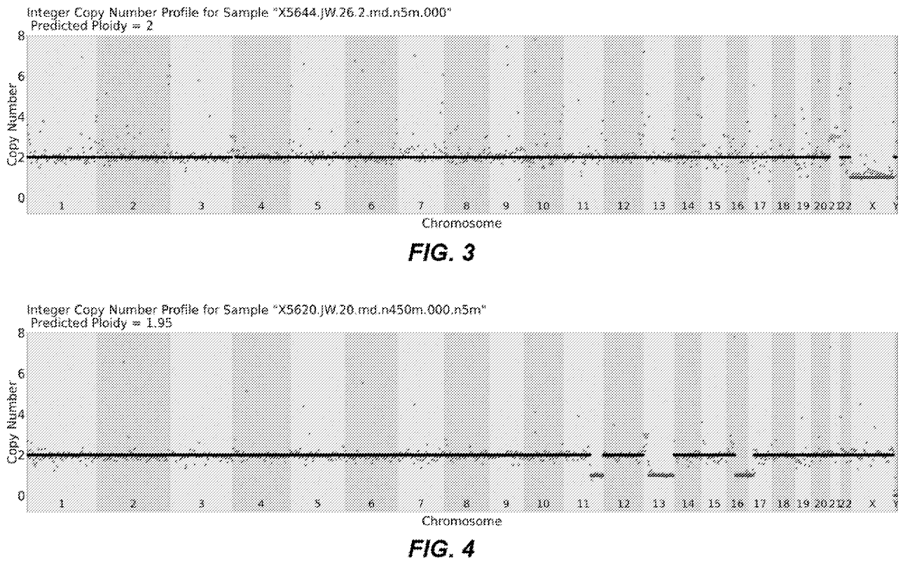

Following the general procedure of Example 1, a biopsy of human embryo was performed following in vitro fertilization (IVF) when the embryo was approximately 5-7 days post-retrieval. Embryo biopsy material was placed into dry, 0.25 mL PCR tubes or placed into PCR tubes containing 1 μL of Cell Lysis Buffer. Each embryo biopsy sample containing between 1 and approximately 6-8 intact cells was subjected to the PTA reaction, and the resulting libraries were sequenced. CNV analysis of the cells was capable of detecting chromosomal disorders, such as trisomy on chromosome 21 (FIG. 3).

### Example 3: SNV Analysis with PTA

Following the general procedure of Example 1, resected breast tumors, some with matched tumor normal samples, were obtained, and single cells subjected to the PTA workflow. Analysis for copy number variation was conducted which identified abnormalities at chromosomes 11, 13, 16, and 17 (FIG. 4). For the same cells, SNV determination was also made (Table 1). All samples were joint-genotyped.

TABLE 1
SNP Counts

| Sample Name | snp_count |
|---|---|
| 5620-JW-1 | 3217840 |
| 5620-JW-2 | 4390344 |
| 5620-JW-3 | 4404955 |
| 5620-JW-4 | 4412810 |
| 5620-JW-5 | 4362113 |
| 5620-JW-6 | 4660197 |
| 5620-JW-7 | 3266906 |
| 5620-JW-8 | 4476649 |
| 5620-JW-9 | 2835066 |
| 5620-JW-10 | 4422578 |
| 5620-JW-11 | 4598355 |
| 5620-JW-12 | 4382265 |
| 5620-JW-13 | 4389891 |
| 5620-JW-14 | 4405770 |
| 5620-JW-15 | 4253536 |
| 5620-JW-16 | 4369933 |
| 5620-JW-17 | 4398855 |
| 5620-JW-18 | 4294615 |
| 5620-JW-19 | 4413475 |
| 5620-JW-20 | 4375069 |
| 5620-JW-21 | 3876068 |
| 5620-JW-22 | 4440532 |
| 5620-JW-23 | 4502563 |
| 5620-JW-24 | 4234510 |
| 5620-JW-25 | 4430321 |
| 5620-JW-26 | 4452879 |
| 5620-JW-27 | 4537440 |
| 5620-JW-28 | 2196647 |
| 5620-JW-29 | 4523231 |
| 5620-JW-30 | 4497218 |
| 5620-JW-31 | 4482111 |

### Example 4: Single Workflow Analysis for PGT

Embryonic cells (5-10) are obtained from a 5 to 7 day old blastocyst generated from IVF, and subjected to the general procedures of Example 1. Each sample is isolated and amplified using PTA to generate libraries, sequencing adapters added, and the libraries are sequenced. From the sequencing results, aneuploidy, monogenic disorders, and structural rearrangements are determined. A single workflow provides information from each individual biopsy sample which is normally only obtainable from separate PGT-A, PGT-M, and PGT-SR testing procedures. Such information may be used to rank order embryos for elective single embryo transfer (e.g., embryos determined to have a euploid chromosome makeup, or cells with the lowest mosaicism rate if no euploid embryos are available and/or absence of fetal genetic abnormalities either known ahead of testing or determined during embryonic testing).

### Example 5: Single Workflow Analysis for NIPT

Following the general procedure of Example 1, a sample, comprising as few as a single cell or up to at least ten(s) of cells are obtained from a pregnant subject using various collection methods. In some instances, the sample is obtained as early as 5 weeks after pregnancy. Fetal cells are selectively stained using either fluorescent or non-fluorescent antibodies specific for inter- and extra-cellular markers. In one embodiment, the antibody is labeled with a magnetic nanoparticle, and fetal cells are separated from maternal cells with a magnet. In another embodiment, the antibody comprises a visually detectable label, and fetal cells are sorted from maternal cells using various methods. After isolation of fetal cells, each sample is subjected to the PTA workflow, sequenced, and analyzed for aneuploidy, monogenic disorders, and structural rearrangements. A single workflow provides genetic information from each individual sample which is normally only obtainable through multiple diagnostic techniques (e.g., blood sample/cell-free fetal DNA, amniocentesis, or CVS (chorionic villus sampling)). If IVF was used to create the embryo, optionally these results are compared with results obtained using PTA from the same embryo prior to embryo transfer.

### Example 6: Primary Template Amplification of Fetal Cells

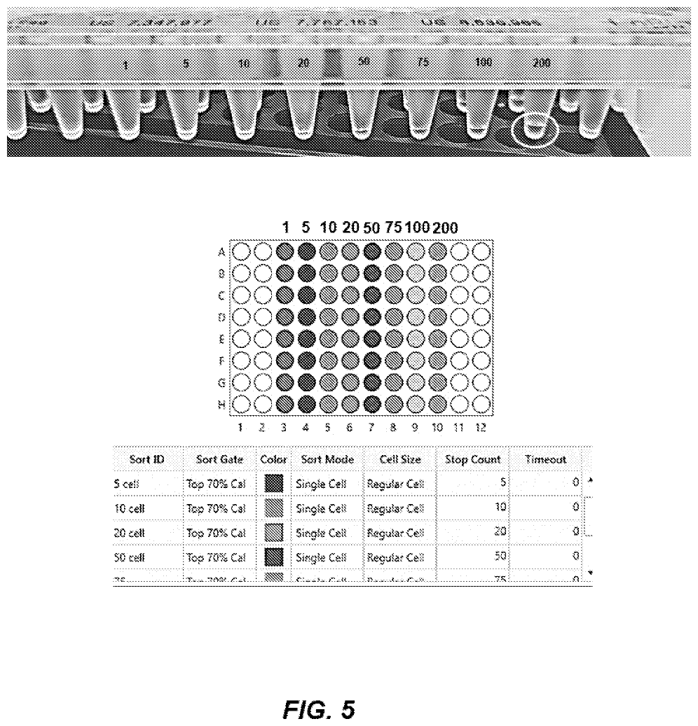

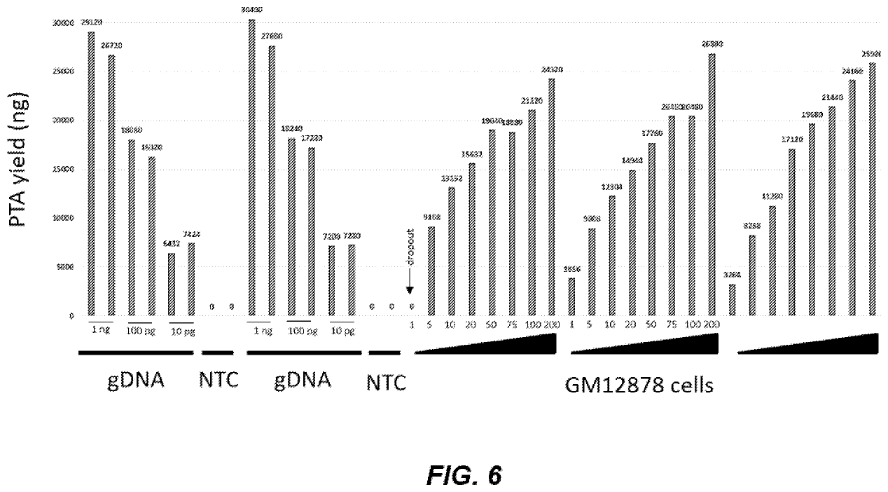

Single cells from cell line NA12878 (HG001) were captured and collected into individual PCR tubes. Tubes containing either 1, 5, 10, 20, 50, 75, 100 or 200 cells were captured in wells, as depicted in FIG. 5. Wells with more cells had a greater volume. Genomic DNA was isolated and the DNA was amplified using PTA. A 100 μL total reaction volume was used with a 25 μL sample input volume. The PTA yield is depicted in FIG. 6. gDNA was used as controls. PTA amplification produced increasing yields of DNA with the increasing number of cells, despite the differences in initial sample volume.

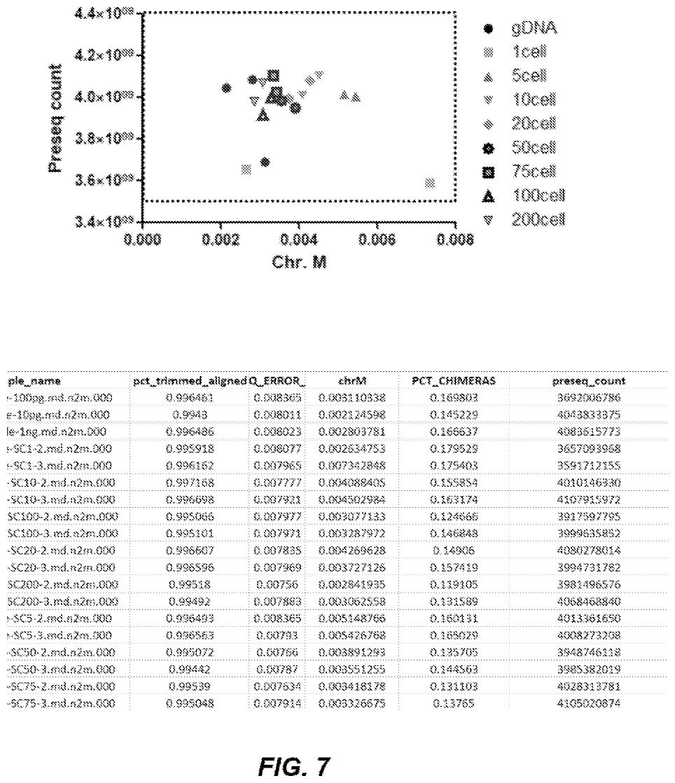

The PTA products were analyzed for quality control. All samples had a preseq count greater than 3.5 E9 and less than 1% Chr.M, as depicted in FIG. 7, indicating that amplification was sufficient for deep sequencing.

### Example 7: Primary Template Amplification of Fetal Cells

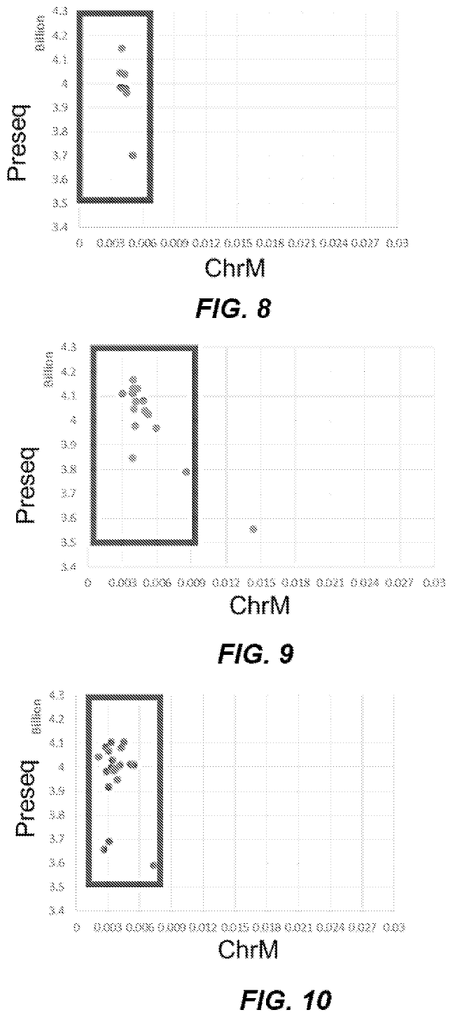

Fetal cells were isolated. Genomic DNA was amplified using PTA. The PTA products were assessed for quality control. Results are depicted in FIGS. 8-10. Samples with a preseq count greater than 3.5 E9 and less than 1% Chr.M were sufficient for deep sequence. High quality samples are indicated with a box. Most samples showed quality values consistent with a quality sufficient for deep sequencing.

### Example 8: Sequencing Hoescht-Stained Cells

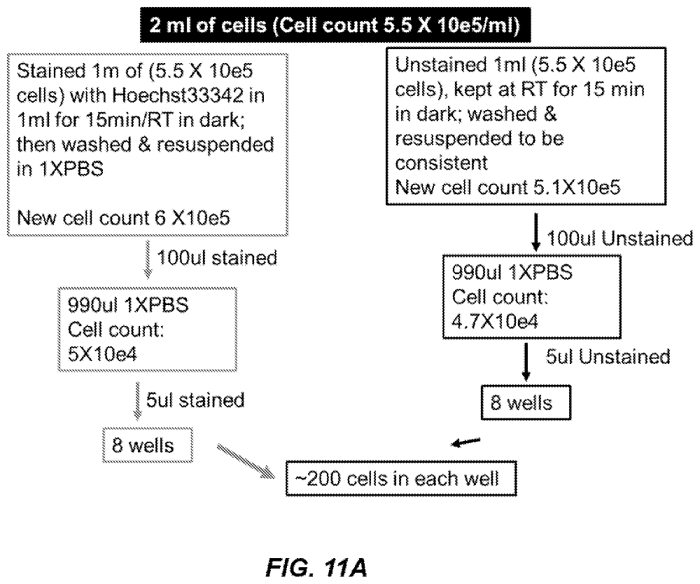

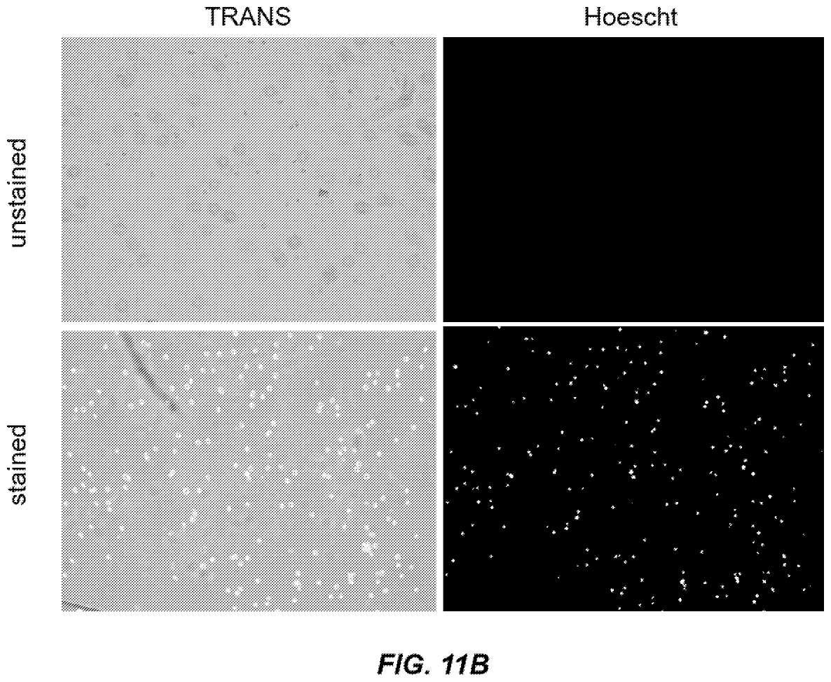

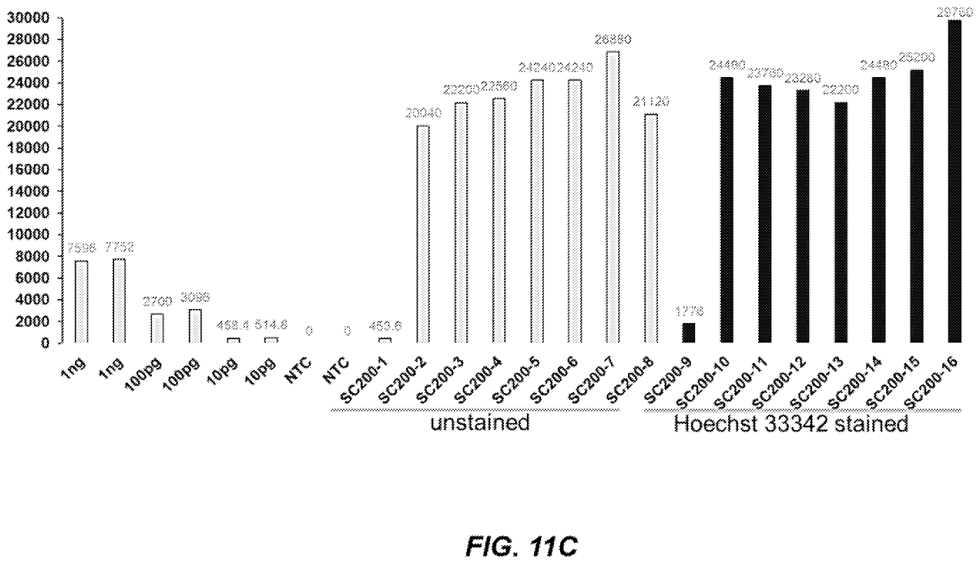

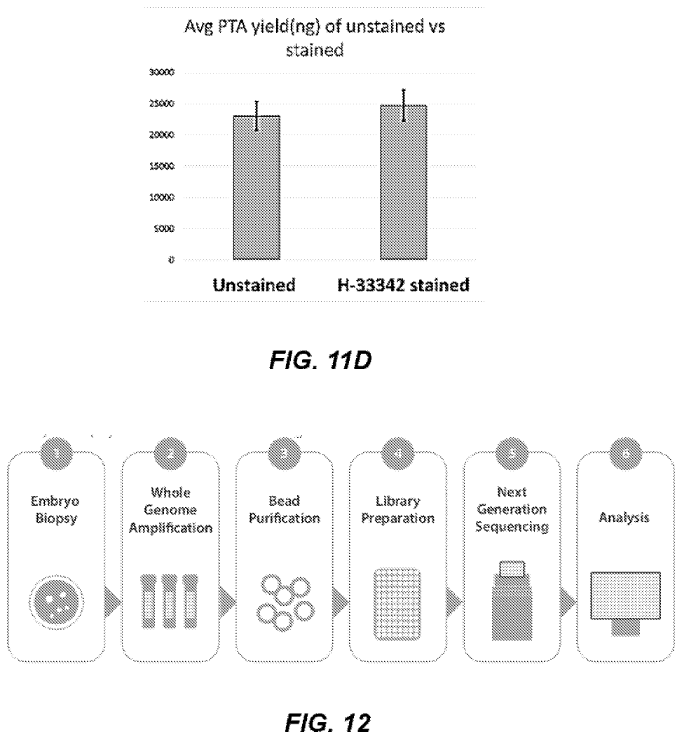

Cells are stained with Hoescht3342 according to the protocol in FIG. 11A. Stained and unstained cells are depicted in FIG. 11B. PTA amplification was performed on both the unstained and stained cells. Both the stained and unstained cells produced similar yields of DNA post-reaction, as depicted in FIGS. 11C-11D. As depicted in Table 2, the initial quality analysis revealed sequencing metrics sufficient for deep sequencing.

TABLE 2
Sequencing metrics

| sample_name | preseq_count | chrM | PCT_CHIMERAS | pct_trimmed_aligned | PF_HQ_ERROR_RATE | PCT_EXC_DUPE |
|---|---|---|---|---|---|---|
| Hoechst-200C2 | 3,901,173,355.00 | 0.005 | 0.1084 | 0.9969 | 0.0091 | 0 |
| Hoechst-200C3 | 3,950,469,840.00 | 0.0045 | 0.1129 | 0.997 | 0.0091 | 0 |
| Hoechst-200C4 | 3,913,134,260.00 | 0.0046 | 0.1104 | 0.9969 | 0.0093 | 0 |
| Hoechst-200C5 | 3,935,345,952.00 | 0.0051 | 0.1138 | 0.996 | 0.0094 | 0 |
| Hoechst-200C6 | 3,870,577,332.00 | 0.005 | 0.1083 | 0.9966 | 0.0091 | 0 |
| Hoechst-200C7 | 3,963,626,368.00 | 0.0047 | 0.1102 | 0.9963 | 0.0099 | 0 |
| Hoechst-200C8 | 3,904,149,918.00 | 0.0051 | 0.1161 | 0.9969 | 0.0091 | 0 |
| nostain100pg | 2,890,292,733.00 | 0.0065 | 0.1754 | 0.9964 | 0.0093 | 0 |
| nostain200C2 | 3,881,152,537.00 | 0.0053 | 0.1209 | 0.9964 | 0.0092 | 0 |
| nostain200C3 | 3,905,017,778.00 | 0.0053 | 0.1163 | 0.9966 | 0.0092 | 0 |
| nostain200C4 | 3,942,442,311.00 | 0.0045 | 0.1162 | 0.9959 | 0.0091 | 0 |
| nostain200C5 | 3,917,385,080.00 | 0.0048 | 0.1083 | 0.9964 | 0.0093 | 0 |
| nostain200C6 | 3,858,069,533.00 | 0.0051 | 0.1145 | 0.9963 | 0.0091 | 0 |
| nostain200C7 | 3,918,680,877.00 | 0.0049 | 0.109 | 0.9968 | 0.0092 | 0 |
| nostain200C8 | 3,936,702,505.00 | 0.0056 | 0.0924 | 0.9973 | 0.0088 | 0 |

### Example 9: A Fully Integrated Workflow for Complete Analysis of Human Embryos for Aneuploidy and Monogenic Disease

All forms of PGT generally require a biopsy of 4-6 cells from the growing embryo followed by genome amplification to create enough DNA to analyze for PGT. These methods include such approaches as multiple displacement amplification (MDA), PicoPlex™, and MALBEC™. Here we detail a new technology, (PTA). The PTA platform allows for enrichment of cellular genomes. By limiting product amplification bias and error propagation, PTA enabled highly accurate whole-genome and targeted analysis of a single cell.

Described here is a workflow that allowed the analysis of gross chromosomal aneuploidy errors (PGT-A) simultaneously with comprehensive monogenic genetic disorder single nucleotide variation analysis (PGT-M), along with structural chromosome rearrangements (PGT-SR).

This fully-integrated workflow (FIG. 12) highlights the steps; from embryo biopsy at the IVF center through whole genome amplification (WGA), library preparation, sequencing and analysis. The described methods of PTA-amplification combined PGT-A, PFT-SR and PGT-M in a single PGT workflow from the biopsy of a single embryo. This allowed for whole genome and/or specific panels for known inherited mutations to be leveraged. Due to the completeness of genome coverage by Primary Template-directed Amplification (PTA), there was no need for splitting samples or multiple workflows. The workflow made this possible from a 4-6 cell embryo biopsy down to a single blastomere.

The three classes of preimplantation genetic testing (PGT) include:

Aneuploidy (PGT-A): In PGT-A, each sample was tested for chromosome gains (trisomy) and losses (monosomy) of all or part of each chromosome, including trisomy 21 (Down syndrome), and trisomy 16 (leading to miscarriage.

Monogenic Disease (PGT-M): In PGT-M, each sample was tested for specific inherited monogenic mutations that lead to disease in humans, such as cystic fibrosis (CF) or Huntington disease (HD) 4.

Structural Chromosome Rearrangements (PGT-SR): In PGT-SR, samples were tested for known structural chromosome rearrangement or chromosome translocation. People with structural chromosome rearrangements typically have no signs or symptoms. However, when they go through meiosis, errors during chromosome alignment cause abnormal gametes with unbalanced chromosomes to be created. PGT-SR allows for selective transfer of normal/balanced embryos and higher chances of delivery following PGT-SR5.

### Initial Validation of PTA for PGT

42 embryo samples donated for research at a local IVF center were biopsied and the cells placed in 0.2 ml Eppendorf tubes in 1×PBS and shipped on dry ice for testing. Following receipt in the lab, the samples were processed through the workflow using the initial biopsy tube, followed by library preparation and sequencing on Illumina platforms. Analysis of each individual embryo sample can be extended beyond conventional PGT-A/PGT-M/PGT-SR to delve deeper into the genetics and potentially genomics of embryos assessed during an IVF cycle.

### DNA Assessment Following PTA Yield and Genomic Coverage

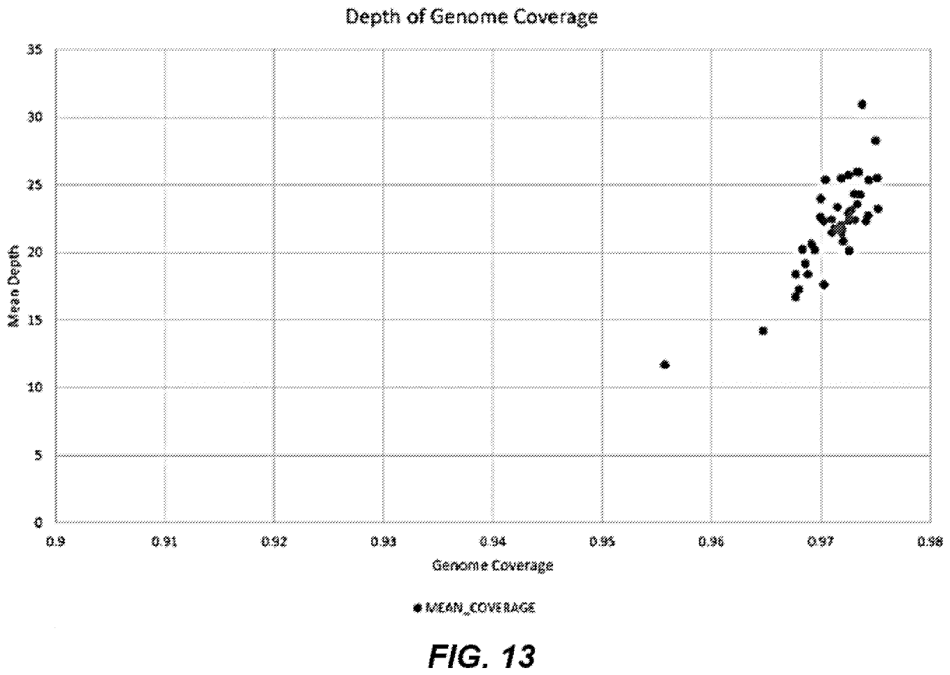

Cells from control and embryo samples were taken through the workflow. Yields for all specimens were as expected for control cells, NTC (No Template Control) negative samples, and embryo samples. At this point, each sample went through library preparation and whole genome sequencing. Following sequencing, the overall average depth of the genome was reported to evaluate ability to enable multiple classes of PGT. Well over 95% of the genome was covered across all 42 embryo samples and the target of 20× depth for variant detection was achieved for the vast majority of the samples (FIG. 13). The majority of embryos tested had greater than 15× depth across over 96% of the genome.

### [PGT-M] Single Nucleotide Variation

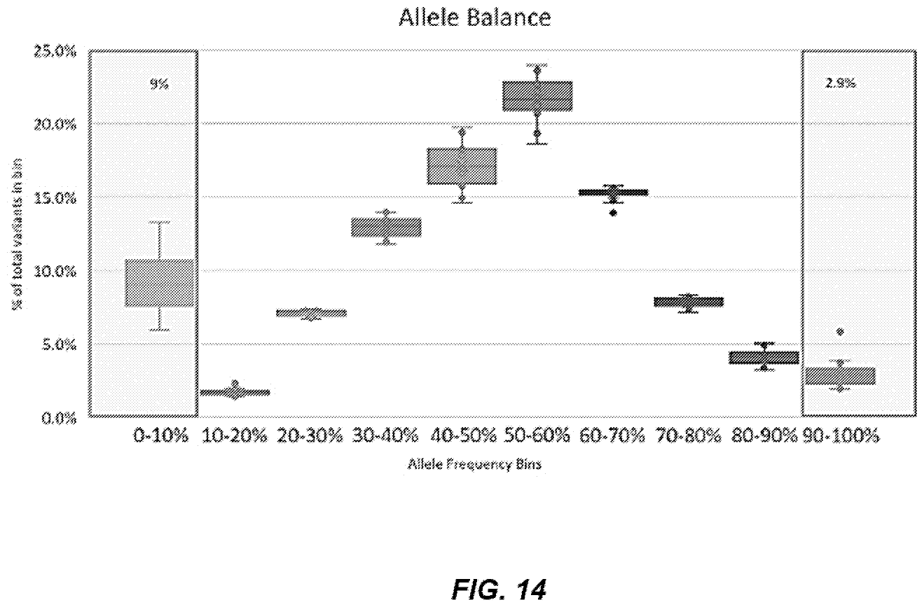

Other technologies for PGT-M requires case-specific workflows in the laboratory that require multiple workflows/platforms (i.e. NGS+PCR or SNP array+PCR) and generally requires splitting of the sample sometime during WGA to aid in creating enough coverage to allow for CNV analysis (PGT-A) while also deeply sequencing in and around the gene(s) of interest for PGT-M. In addition to the robust, wide genome coverage required for assessment of copy number variation, the PTA Workflow allowed for the ability to report changes in alleles across the genome. Table 3 highlights the genomic representation observed using the PTA workflow, while FIG. 14 demonstrates the recovery of both alleles.

TABLE 3
Performance characteristics for PTA workflow

| Genome | | | Allelic | Allelic |
|---|---|---|---|---|
| Coverage | Sensitivity | Precision | Balance (@20x) | Balance |
| 96% | 93.5% | 99.2% | 88.1% | 98.1% |

Cells isolated from NA12878 (HG001) were leveraged to characterize workflow performance. Values represent averages across all replicates in an internal study. Additional sequencing was performed to measure allelic balance up to 40× mean depth. Allelic balance is a summary of all heterozygous loci that were called as heterozygous in our pipeline.

Allele ratio bins for the embryos sequenced to 20× were compared from genome-wide variants confirmed to be heterozygous. Allele drop out is for variants <10% or >90% and the median value of variants in those bins across all samples are shown.

To better understand the genomic coverage, embryos were assessed for the uniformity and depth of coverage in 5 example genes that are frequently tested for using PGT-M including BRCA1 (Breast Cancer), CFTR (Cystic Fibrosis), DMD (Duchenne Muscular Dystrophy) and HTT (Huntington Disease). In these specific genes, coverage of the genome was assessed that would allow for analysis of typical mutations in each gene along with single nucleotide polymorphisms (SNPs) to add assurance (ADO, contamination, PA, etc.) to their testing (Table 4). Gene results were summarized across all coding regions of a gene. Depth of 4 provides power to call heterozygous variants. Uniformity of coverage is the percent of gene regions where depth is within 20% of the mean.

TABLE 4
Performance of Typical genes involved in PGT-M testing.

| | Coverage | | Mean | Uniformity of |
|---|---|---|---|---|
| Gene | (%) | Coverage> | Depth | Coverage |
| BRCA1 | 100 | 100 | 32.1 | 36.43 |
| CFTR | 100 | 99.98 | 25.6 | 28.98 |
| DMD | 99.98 | 99.26 | 17.8 | 33.99 |
| HTT | 98.36 | 94.76 | 23.5 | 29.29 |

### PGT-A (Copy Number Variation Detection)

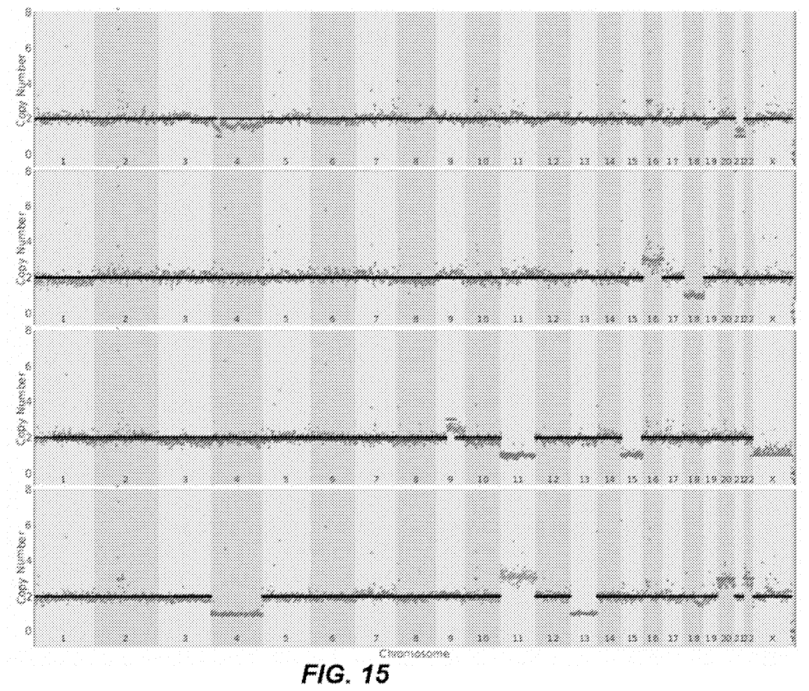

For PGT-A, copy number variation (CNV) is typically limited to assessment of chromosome changes no smaller than 7-10 MB gains/losses. Therefore any gain/loss smaller than this window were not called or reported. Analysis of CNV relies on wide genome coverage and can be affected by sequencing parameters (single end vs. paired end, number of sequencing cycles, etc.). To evaluate the performance of the PTA workflow, we assessed individual embryo biopsy samples with known aneuploidy using PTA chemistry and an optimized, 1 Mb bin CNV calling algorithm (FIG. 15). The PTA workflow characterizes known aneuploidy with a resolution of 5-7 Mb, consistent with industry standard.

### CONCLUSIONS

This workflow showed completeness of coverage and genome depth on biopsied embryos. This allowed for testing embryos for PGT-A, PGT-M and PGT-SR from a single biopsy without the need for additional platforms, software, amplification or sample splitting.

The examples described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of embryonic nucleic acid sample preparation useful for determining the presence of fetal genetic abnormalities comprising:
a. isolating nucleic acids from at least one embryonic cell;
b. subjecting the nucleic acids to a sample workflow; and
c. determining if the embryonic cell comprises at least two fetal genetic abnormalities by analyzing the nucleic acids from the sample workflow, wherein the fetal genetic abnormalities comprise:
i. at least one copy number variation; and
ii. at least one single nucleotide variant.

2. The method of claim 1, wherein the embryonic cell comprises a preimplantation embryonic cell, a blastocyte cell, blastomere cell, a cell obtained from the trophectoderm, a placental cell, or a cell derived from extra-embryonic membranes.

3. The method of claim 1 or 2, wherein the embryonic cell comprises a preimplantation embryonic cell.

4. The method of any one of claims 1-3, wherein the fetal genetic abnormality comprises two or more of aneuploidy, monogenic disorders, and structural rearrangements.

5. The method of any one of claims 1-4, wherein the fetal genetic abnormality comprises aneuploidy, monogenic disorders, and structural rearrangements.

6. The method of any one of claims 1-5, wherein determining comprises obtaining information on fetal genetic abnormalities identifiable by PGT-A, PGT-M, or PGT-SR testing.

7. The method of any one of claims 1-6, wherein the genetic abnormality comprises phenylketonuria (PKU), sickle-cell anemia, Beta Thalassemia, Tay-Sachs disease, Sandhoff disease, or cystic fibrosis (CF).

8. The method of any one of claims 1-6 wherein the genetic abnormality comprises achondroplasia, congenital adrenal hyperplasia, Cystic fibrosis, Down syndrome, fragile XD syndrome, Hemophilia A, Huntington's disease, Muscular dystrophy, Polycystic kidney disease, Sickle cell disease, Tay-Sachs disease, trisomy 21, trisomy 18, trisomy 13, Turner syndrome, spina bifida, anencephaly, or Thalassemia.

9. The method of any one of claim 4, wherein the aneuploidy comprises monosomy, trisomy, triploidy, deletions, duplications, or uniparental disomy.

10. The method of claim 9, wherein the uniparental disomy occurs at least in four chromosomes.

11. The method of claim 9, wherein the uniparental disomy occurs at chromosomes 6, 7, 11, 14, or 15.

12. The method of any one of claims 1-11, wherein the fetal genetic abnormality comprises an insertion, deletion or duplication.

13. The method of claim 12, wherein the insertion, deletion or duplication is at least 5% of the total chromosome length.

14. The method of claim 12 or 13, wherein the insertion, deletion or duplication is less than 15% of the total chromosome length.

15. The method of any one of claims 1-14, wherein the method further comprises obtaining the cell from at least a 5 day old blastocyte.

16. The method of any one of claims 1-15, comprising at least 4 embryonic cells.

17. The method of any one of claims 1-16, wherein a fetal genetic abnormality is detected in no more than 30% of the embryonic cells.

18. The method of any one of claims 1-17, wherein a fetal genetic abnormality is detected in 30%-100% of the embryonic cells.

19. The method of any one of claims 1-18, wherein the method further comprises obtaining the embryonic cell from a location proximal to an external os of a uterine cervix or anywhere within the vaginal canal of a subject.

20. The method of any one of claims 1-19, wherein the method further comprises obtaining the embryonic cell from a Pap smear.

21. The method of any one of claims 1-20, wherein the embryonic cell is human.

22. The method of any one of claims 1-21, comprising 6-200 embryonic cells.

23. The method of claim 22, further comprising measuring a level of mosaicism for the embryonic cells.

24. The method of any one of claims 1-23, further comprising establishing the presence or absence of sex chromosomes in the embryonic cell.

25. The method of any one of claims 1-24, wherein the embryonic cell is a preimplantation embryonic cell from an embryo, and the method further comprises implanting the embryo in a female.

26. The method of any one of claims 1-25, wherein the fetal genetic abnormalities are determined without a blood or saliva test.

27. The method of any one of claims 1-26, wherein the fetal genetic abnormalities are determined without amniocentesis, chorionic villus sampling, or Percutaneous umbilical blood sampling.

28. The method of any one of claims 1-27, wherein determining comprises sequencing the nucleic acids.

29. The method of claim 28, wherein sequencing comprises Sanger sequencing, next generation sequencing, single-molecule real-time sequencing, Polony sequencing, sequencing by synthesis, sequencing by ligation, reversible terminator sequencing, proton detection sequencing, ion semiconductor sequencing, nanopore sequencing, electronic sequencing, pyrosequencing, Maxam-Gilbert sequencing, chain termination sequencing, or +S sequencing.

30. The method of claim 28 or 29, wherein the method further comprises exome capture prior to sequencing.

31. The method of any one of claims 1-30, wherein the sample workflow comprises:
contacting the nucleic acids with at least one amplification primer, at least one nucleic acid polymerase, and a mixture of nucleotides, wherein the mixture of nucleotides comprises at least one terminator nucleotide which terminates nucleic acid replication by the polymerase, and
amplifying at least some of the nucleic acids to generate a plurality of terminated amplification products, wherein the replication proceeds by strand displacement replication.

32. A method of embryonic nucleic acid sample preparation useful for determining the presence of fetal genetic abnormalities comprising:
a. isolating at least one embryonic cell from a plurality of cells, wherein the plurality of cells comprises fetal and maternal cells;
b. isolating nucleic acids from the at least one embryonic cell;
c. contacting the nucleic acids with at least one amplification primer, at least one nucleic acid polymerase, and a mixture of nucleotides, wherein the mixture of nucleotides comprises at least one terminator nucleotide which terminates nucleic acid replication by the polymerase;
d. amplifying at least some of the nucleic acids to generate a plurality of terminated amplification products, wherein the replication proceeds by strand displacement replication; and
e. determining if the embryonic cell comprises one or more genetic abnormalities by analyzing the terminated amplification products.

33. The method of claim 32, wherein the embryonic cell is isolated from the plurality of cells on a surface.

34. The method of claim 32 or 33, wherein the fetal cells are obtained from one or more of the trophectoderm, placenta, or extra-embryonic membranes.

35. The method of any one of claims 32-34, wherein the embryonic cell is isolated by an automated robotic device.

36. The method of claim 35, wherein the robotic device comprises a capillary fitting.

37. The method of claim 32, wherein the embryonic cell is uniquely identified from other non-embryonic cells.

38. The method of claim 32, wherein the embryonic cell is uniquely identified from other non-embryonic cells by labeling.

39. The method of claim 38, wherein labeling comprises contacting the plurality of cells with an antibody.

40. The method of claim 39, wherein the antibody is configured to bind selectively to non-fetal cells.

41. The method of claim 39, wherein the antibody is configured to bind selectively to fetal cells.

42. The method of claim 39, wherein the antibody is configured to bind to HLA-G or βhCG.

43. The method of claim 38, wherein labeling comprises Hoescht staining.

44. The method of any one of claims 32-42, wherein isolating comprises FACS sorting.

45. The method of claim 39, wherein the antibody comprises a magnetic nanoparticle.

46. The method of any one of claims 32-45, wherein the embryonic cell is removed as early as 5 weeks after pregnancy.

47. The method of any one of claims 32-45, wherein the embryonic cell is removed as early as 8 weeks after pregnancy.

48. The method of any one of claims 32-47, wherein determining comprises in-silico removal of maternal nucleic acid sequences.

49. The method of any one of claims 32-48, wherein the terminator is an irreversible terminator.

50. The method of any one of claims 32-48, wherein the terminator nucleotide is selected from the group consisting of nucleotides with modification to the alpha group, C3 spacer nucleotides, locked nucleic acids (LNA), inverted nucleic acids, 2′ fluoro nucleotides, 3′ phosphorylated nucleotides, 2′-O-Methyl modified nucleotides, and trans nucleic acids.

51. The method of claim 50, wherein the nucleotides with modification to the alpha group are alpha-thio dideoxynucleotides.

52. The method of any one of claims 32-51, wherein the terminator nucleotide comprises modifications of the r group of the 3′ carbon of the deoxyribose.

53. The method of any one of claims 32-52, wherein the terminator nucleotide is selected from the group consisting of dideoxynucleotides, inverted dideoxynucleotides, 3′ biotinylated nucleotides, 3′ amino nucleotides, 3′-phosphorylated nucleotides, 3′-O-methyl nucleotides, 3′ carbon spacer nucleotides including 3′ C3 spacer nucleotides, 3′ C18 nucleotides, 3′ Hexanediol spacer nucleotides, acyclonucleotides, and combinations thereof.

54. The method of any one of claims 32-53, wherein the plurality of terminated amplification products comprise an average of 1000-2000 bases in length.

55. The method of any one of claims 32-54, wherein at least some of the amplification products comprise a cell barcode or a sample barcode.

56. The method of any one of claims 32-55, wherein amplification occurs for at least five cycles.

57. A method of embryonic nucleic acid sample preparation useful for determining the presence or absence of fetal genetic abnormalities comprising:
a. obtaining a plurality of embryonic cells, wherein the plurality of embryonic cells comprises between 2 and 200 embryonic cells;
b. isolating nucleic acids from the plurality of embryonic cells;
c. contacting the nucleic acids with at least one amplification primer, at least one nucleic acid polymerase, and a mixture of nucleotides, wherein the mixture of nucleotides comprises at least one terminator nucleotide which terminates nucleic acid replication by the polymerase;
d. amplifying at least some of the nucleic acids to generate a plurality of terminated amplification products, wherein the replication proceeds by strand displacement replication; and
e. determining if the plurality of embryonic cells comprises one or more genetic abnormalities by analyzing the terminated amplification products.

58. The method of claim 32 or 33, wherein the fetal cells are obtained from one or more of the trophectoderm, placenta, or extra-embryonic membranes.
